(19) United States
(12) Patent Application Publication
(10) Pub. No.: US 2026/0099665 A1
(43) Pub. Date: Apr. 9, 2026

(54) PLATFORM FOR MEDICAL PRODUCT DEVELOPMENT AND COMPLIANCE SUBMISSIONS

(71) Applicant: Accelidea, Inc., Quincy, MA (US)

(72) Inventor: Rose Yi Huang, Quincy, MA (US)

(21) Appl. No.: 19/353,057

(22) Filed: Oct. 8, 2025

Related U.S. Application Data
(60) Provisional application No. 63/705,323, filed on Oct. 9, 2024.

(51) Int. Cl. G06F 40/166; G06F 40/197; G16H 50/30

(52) U.S. Cl. CPC G06F 40/166; G06F 40/197; G16H 50/30

(57) ABSTRACT

A platform for automatic alignment across device design stages in device development includes an integrated interface with modules corresponding to device design stages. An alignment module of the platform includes a workflow associated with a device. The workflow includes documents corresponding to requirements according to regulations or standards associated with a device profile with attributes for the device. The platform receives a selection of a current document associated with attributes and tasks to be performed to complete the current document. The platform receives a design decision including an update to a value of an attribute associated with the current document. The current document is updated with the value, and the design decision is automatically propagating in the workflow. The propagation includes identifying upstream or downstream documents in the workflow with the attribute and updating the attribute in these documents. The design decision is also automatically propagating to the modules.

### CROSS-REFERENCE TO RELATED APPLICATIONS

The present application claims the benefit of U.S. Provisional Patent Application Ser. No. 63/705,323, which was filed on Oct. 9, 2024, titled “Platform for Medical Device Product Development and Certification Submissions”, which is hereby incorporated by reference.

### BACKGROUND

### Technical Field

This application relates generally to digital platforms for product development and compliance, and specifically to a digital platform for medical product development and compliance submissions.

### Background Information

In the rapidly evolving medical technology sector, the distance between a brilliant idea and its market introduction is not just a journey, it's a critical race against time.

Traditional development processes, notorious for inefficiencies, can make this race a costly marathon, draining millions of dollars and delaying life-saving advancements for years.

Yet, even with so many tools at their disposal, cross-functional teams still find themselves mired in manual research, document writing, and data transferring between departments and design stages, each step more time-consuming and costly than the last.

### SUMMARY

An example method for automatic alignment across device design stages in device development, includes: displaying an integrated interface including a plurality of modules corresponding to the device design stages; displaying an interface for an alignment module of the plurality of modules, the alignment module including a workflow associated with a device, the workflow including a plurality of documents corresponding to requirements according to one or more regulations or standards associated with a device profile for the device, the device profile including a plurality of attributes; receiving a selection of a current document of the plurality of document, where the current document is associated with one or more of the plurality of attributes and one or more tasks to be performed to complete the current document; receiving a design decision including at least an update to a value of an attribute associated with the current document; updating the attribute associated with the current document with the value; automatically propagating the design decision in the workflow, including identifying one or more documents upstream or downstream from the current document in the workflow, the one or more documents including the attribute, and updating the value of the attribute in the one or more first documents; and automatically propagating the design decision to one or more other modules of the plurality of modules.

An example system, includes: one or more memories; and one or more processors communicatively coupled to the one or more memories, the one or more processors being configured to: display an integrated interface including a plurality of modules corresponding to the device design stages; display an interface for an alignment module of the plurality of modules, the alignment module including a workflow associated with a device under development, the workflow including a plurality of documents corresponding to requirements according to one or more regulations or standards associated with a device profile for the device, the device profile including a plurality of attributes; receive a selection of a current document of the plurality of document, where the current document is associated with one or more of the plurality of attributes and one or more tasks to be performed to complete the current document; receive a design decision including at least an update to a value of an attribute associated with the current document; update the attribute associated with the current document with the value; automatically propagate the design decision in the workflow, including identifying one or more documents upstream or downstream from the current document in the workflow, the one or more documents including the attribute, and updating the value of the attribute in the one or more first documents; and automatically propagate the design decision to one or more other modules of the plurality of modules.

### BRIEF DESCRIPTION OF THE DRAWINGS

The description below refers to the accompanying drawings, of which:

FIG. 1 includes a block diagram illustrating an example computing environment that includes a platform for product development and compliance.

FIG. 2 includes a block diagram illustrating in more detail a product development and compliance platform according to some embodiments.

FIG. 3 a block diagram illustrating in more detail the one or more outputs of the product development and compliance platform.

FIG. 4 includes a flowchart of a method implemented by the automated research module according to some embodiments.

FIG. 5 includes a flowchart of a method implemented by the automatic cross-functional alignment module according to some embodiments.

FIG. 6 includes a flowchart of a method for monitoring and integrating regulation changes according to some embodiments.

FIG. 7 includes a flowchart of a method for automatic end-to-end traceability according to some embodiments.

FIG. 8 includes a flowchart of a method for automatic risk management according to some embodiments.

FIG. 9A includes a flowchart of a method for identifying hazardous situation and foreseeable sequence of events, according to some embodiments.

FIG. 9B includes a flowchart of a method for identifying severity and occurrences of harm, according to some embodiments.

FIG. 9C includes a flowchart of a method for identifying comparable risks within an acceptable level, according to some embodiments.

FIG. 10 includes a flowchart of a method for automatic documentation and real-time impact assessment according to some embodiments.

FIG. 11 includes a flowchart of a method for automatic alignment across design stages in product development according to some embodiments.

FIG. 12 includes a block diagram for a computer system according to some embodiments.

FIG. 13 includes an example interface of an alignment module in the product development portal.

FIG. 14 includes an example interface for a document in the alignment module.

FIG. 15 includes an example interface of the alignment module modified based on an updated attribute.

FIG. 16 includes an example interface of a traceability matrix.

### DETAILED DESCRIPTION OF AN ILLUSTRATIVE

### Embodiment

Techniques are discussed herein for automatic alignment across design stages and processes in product development and compliance. These are examples, and other examples may be implemented. Particular aspects of the subject matter described in this disclosure can be implemented to realize end-to-end automatic alignment across design stages and processes, where updates at any stage are automatically propagated to affected upstream and downstream stages and processes. This allows for significant increases in efficiency and accuracy of the product development and compliance process, which may lead to lower costs. Items and/or techniques described herein may provide one or more of the following capabilities, and possibly one or more other capabilities not mentioned. Other capabilities may be provided and not every implementation according to the disclosure must provide any, let alone all, of the capabilities discussed.

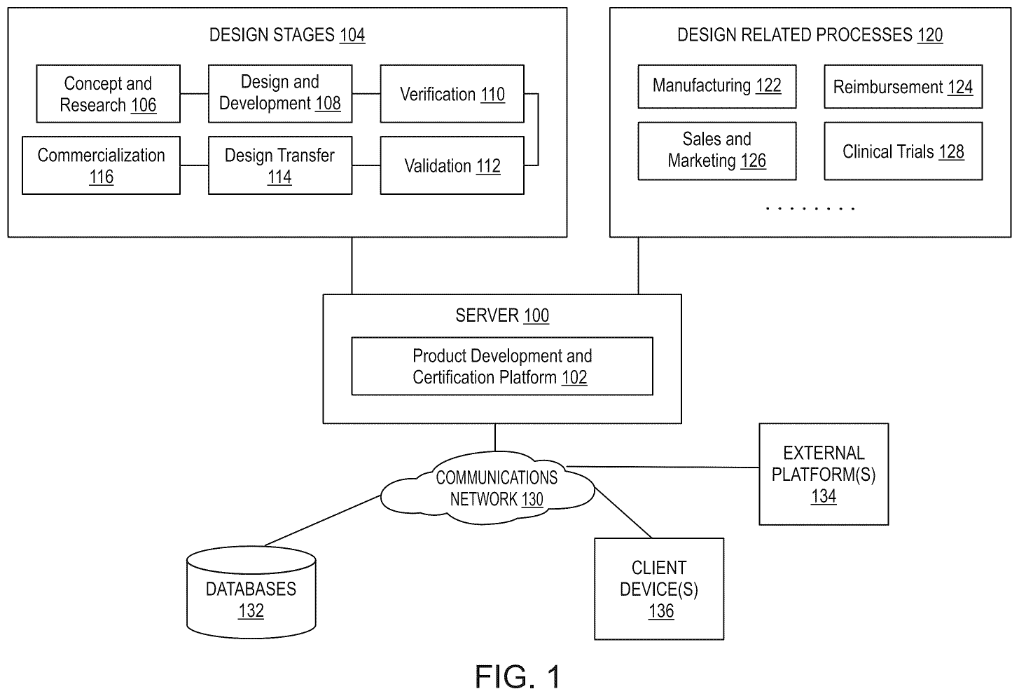

FIG. 1 includes a block diagram illustrating an example computing environment that includes a platform for product development and compliance. The environment includes a server 100 configured to implement a hardware or software product development and compliance platform 102 (“platform”). The platform 102 may be configured to communicate with one or more client devices 136 and one or more external platforms 134 via a communications network 130. The platform 102 may further be configured with access to one or more databases 132 for the storage of data generated by the platform 102 and/or retrieved from one or more external platforms 134, as described further herein. One or more of the databases 132 may be under the control of the platform 102 or shared control with another party, such as a customer. The platform 102 facilitates automatic (without user interference) alignment across design stages and processes in product development and compliance. For example, the design stages 104 may include a concept and research stage 106, a design and development stage 108, a verification stage 110, a validation stage 112, a design transfer stage 114, and a commercialization stage 116. The concept and research stage 106 may include the conceptualization and discovery of a product, which may involve market research to identify market needs, discovering potential technologies and solutions, and developing design specifications and requirements. The design and development stage 108 may include creating the design inputs (detailed requirements for the product functionality performance and safety), design process, and design outputs (detailed specifications, prototypes, and documentation for the product). The verification stage 110 may include confirming that the product meets the design specifications and requirements. The validation stage 112 may include confirming that the product meets user needs and intended uses in real-world conditions. The design transfer stage 114 may include the transfer from product design to manufacturing, ensuring that the product can be consistently produced to meet the design specifications, regulatory requirements, and safety standards. The commercialization stage 116 may include bringing the product to market after regulatory approval and may include scaling manufacturing, creative marketing and distribution strategies, and post-market surveillance. One of more of the design stages 104 may involve one or more design related processes 120, such as manufacturing processes 122, reimbursement processes 124, sales and marketing processes 126, clinical trials 128, etc.

Although the design stages 104 are illustrated as being linear, the design stages 104 in reality are interrelated in a non-linear fashion, with development and changes in one stage affecting one or more other stages. Development and changes in the design related processes 120 may further result in changes in one or more of the design stages 104 or vice versa. The interconnectedness of the design stages 104, among themselves and/or with the design related processes 120, pose technical challenges in ensuring cross-functional alignment. Because the platform 102 automates alignment across design stages and processes, updates at any stage are automatically propagated to affected upstream and downstream stages and processes, significantly increasing the efficiency and accuracy of the product development and compliance process. This in turn leads to lower costs. The platform 102 directly addresses the technical challenges by automatically propagating design decisions across design stages, automatically assessing the design decisions to ensure compliance with regulations and standards, automatically generating end-to-end traceability as design decisions are made across the design stages and monitoring and integrating changes in regulations and standards and automatically propagating the changes across the design stages. The platform 102 improves the technical field of product development and compliance, and more specifically the product development and compliance for medical devices or medical-related services.

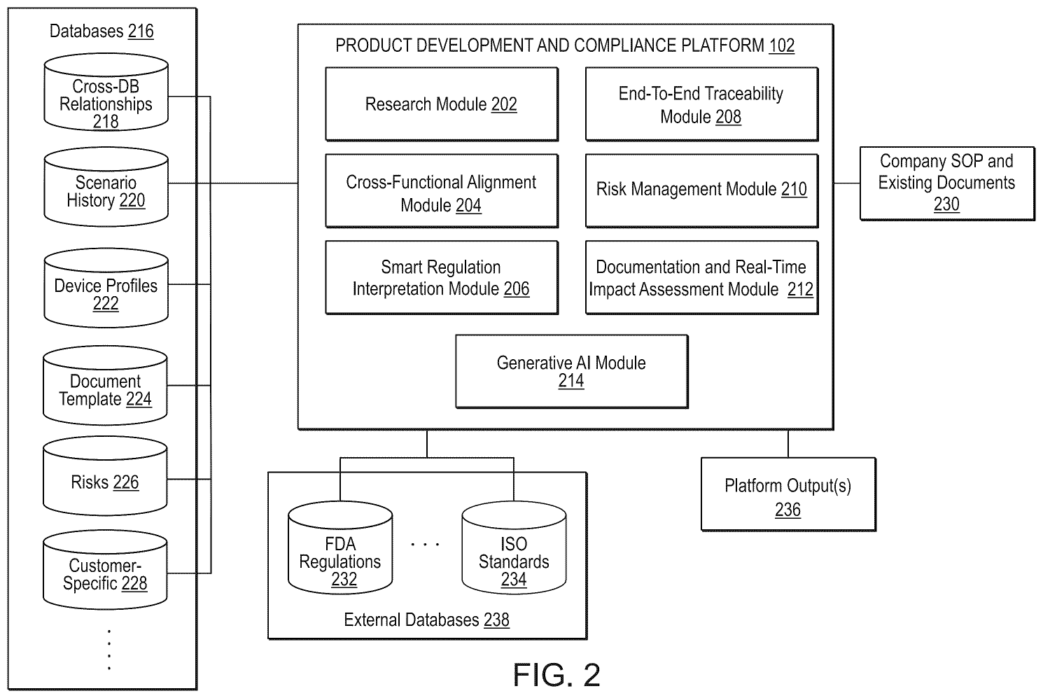

FIG. 2 includes a block diagram illustrating in more detail a product development and compliance platform according to some embodiments. The platform 102 may include an automated research module 202 (“research module”), an automatic cross-functional alignment module 204 (“alignment module”), a smart regulation interpretation module 206 (“interpretation module”), an automatic end-to-end traceability module 208 (“traceability module”), a risk management module 210, an automatic documentation and real-time impact assessment module 212 (“impact assessment module”), and a generative artificial intelligence module 214 (“GenAI module”). The platform 102 is configured with access to a plurality of databases 216. The platform 102 interprets and maintains the relationships between device design parameters within and across various databases and stores the relationships in a database 218 for use throughout the design stages 104 and/or the design related processes 120. A “device design parameter”, as used herein, refers to a device design factor or value applicable to a specific device under development. Categories of device design parameters (or “parameters”) may include regulations, standards, device structure, requirements, specifications, attributes, test cases, labeling, documents related to regulatory submissions, and competitive analyses, as well as others. A “design decision”, as used herein, refers to an update to one or more device design parameters. The databases 216 may include a scenario history database 220 that stores a comprehensive history of scenarios, a device profiles database 222 that stores the attributes of devices being developed, a document templates database 224 for storing templates of documents such as documents required for regulatory submissions, and a risks database 226 for storing hazards, hazardous situations, and harm related to components and other device characteristics. A “scenario”, as used herein, refers to a hypothetical situation or a specific set of circumstances described to demonstrate the safety and effectiveness of a device. The databases 216 may further include customer-specific database(s) 228, where the database(s) 228 are configured with the customer as data owner and database administrator. The platform 102 may also be configured with access to external databases 238, such as Food and Drug Administration (FDA) regulations database 232 and International Organization for Standardization (ISO) standards database 234. These databases are examples only and other databases may be included. In addition, the platform 102 may incorporate any company specific standard operating procedures (SOP) and existing company documents 230. The platform 102 is configured to provide one or more platform outputs 224, which may include various documentations and materials required for a regulatory submission. Through maintaining the relationships between parameters across the databases 216, the platform 102 effectively integrates the information in the various databases and centralizes them into a unified system. A “relationship” represents a connection between parameters. For example, with a medical device, attributes of a drug or device may be interconnected in a structured framework. The relationships between the attributes may demonstrate how the device is developed, manufactured, and interacts with the human body.

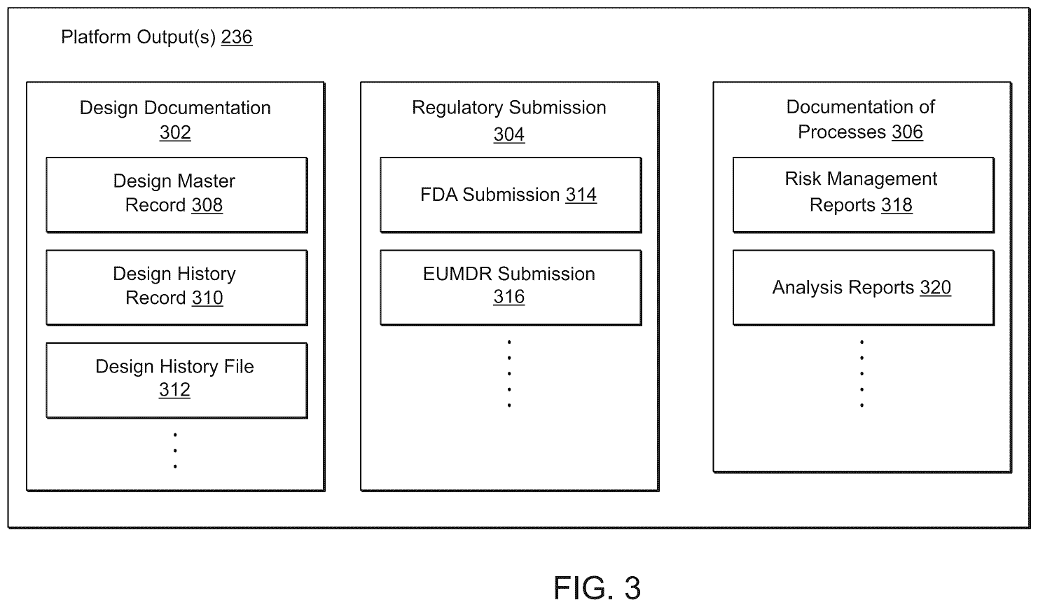

FIG. 3 includes a block diagram illustrating in more detail the one or more outputs of the product development and compliance platform. The outputs 236 may include design documentation 302, regulatory submissions 304, and documentation of processes 306. The design documentation 302 may include documents related to the developments during the design stages 104. For example, the design documentation 302 may include a design master record (DMR) 308, which includes an “instruction manual” for the manufacturing of the medical device. The DMR 308 may include design specifications, the bill of materials (BOMs), production processes, equipment specifications, packaging and labeling specifications, quality assurance (QA) procedures, maintenance and servicing procedures, and acceptance criteria. The design documentation 302 may further include a design history record (DHR) 310, which includes proof that the DMR 308 was compiled correctly. The design history file (DHF) 312 may include design and development plans, user requirements, design inputs, design outputs, design review records, verification and validation (V&V) requirements, design verification results, and change control and corrective and preventive action (CAPA) records. The DHR 312 may include acceptance records, product and component identifiers, material lots, and production records. Other design stage-related documentation may also be included in the design documentation 302. The regulatory submissions 304 may include: FDA submissions 314; European Union Medical Device Regulation (EUMDR) submissions 316; and/or submissions to other regulatory bodies. The FDA submissions 314 may include the DHR 310, the DRM 308, digital data flow (DDF) file, and the medical device file (MDF) according to the corresponding ISO. The EUMDR submissions 316 may include the technical documentation, e.g., the DHF 310, the DMR 308, and post-market documentation. The documentation of processes 306 may include: risk management reports 318, which may include a risk management plan, analysis, evaluation, controls, residual risk evaluation, and review; analysis reports 320, which may detail the methodology, variables, and acceptance criteria for a test, summarize the test results with tables and statistical analyses, and provide a description of the data used; and/or other reports related to the design stages and processes 120.

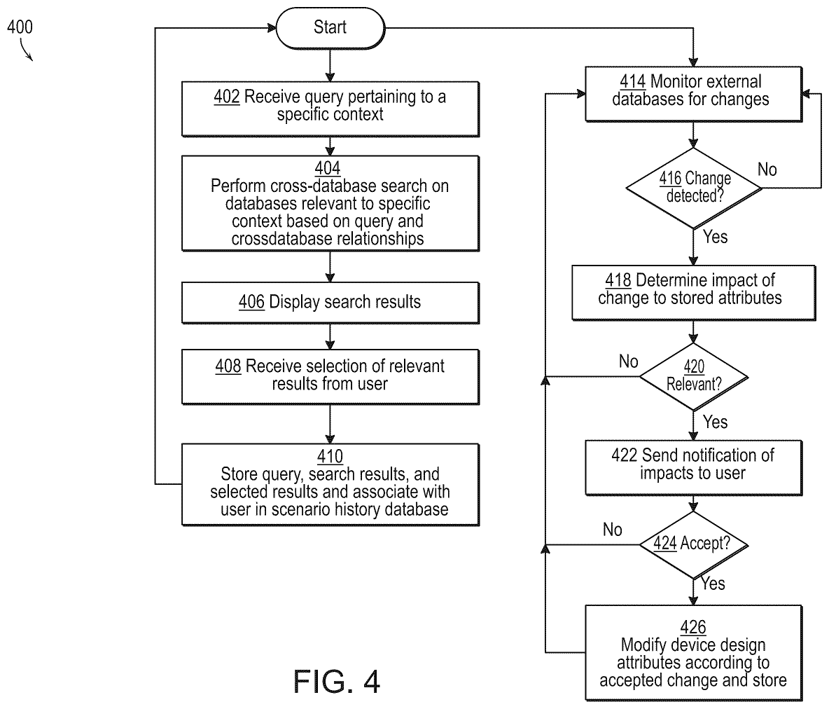

Referring again to FIG. 2, the research module 202 monitors the various databases and automates the backend research process. The research module 202 is further described below with reference to FIG. 4.

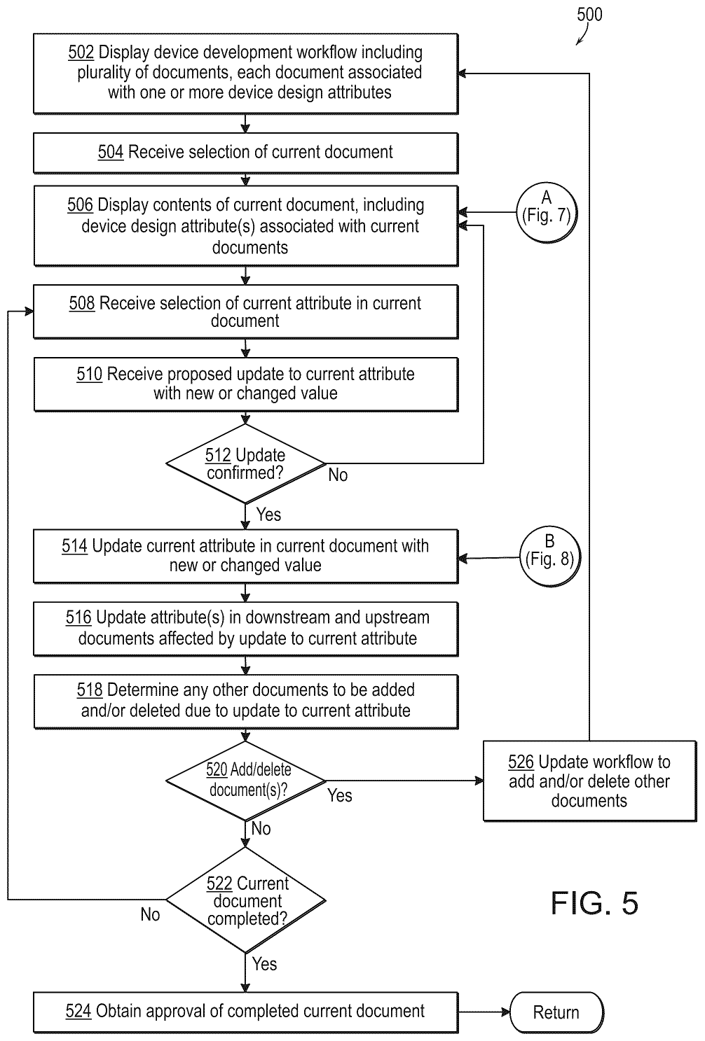

The alignment module 204 provides a dynamic, real-time product development portal with a graphical interface through which a user may interact. The alignment module 204 processes the interactions with the user, facilitates cross-functional alignments, and updates relationships and device attributes based on the user interactions. The alignment module 204 is further described below with reference to FIG. 5.

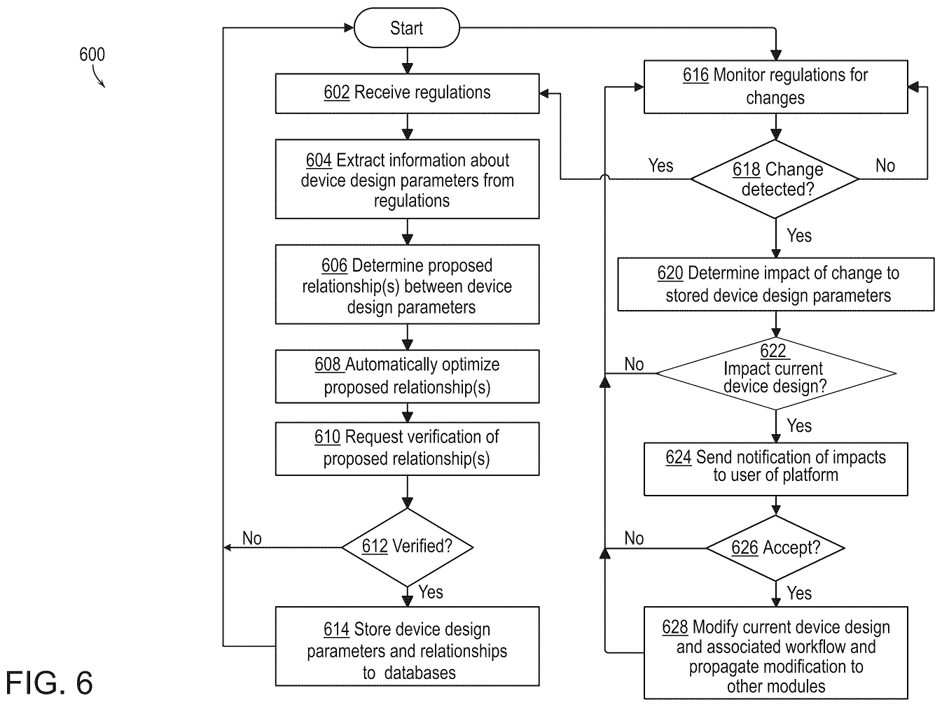

The interpretation module 206 is configured to automatically identify regulations relevant to the device under development and integrates them into the design stages 104. The interpretation module 206 further monitors for changes to regulations and dynamically updates any affected parameters. The interpretation module 206 is further described below with reference to FIG. 6.

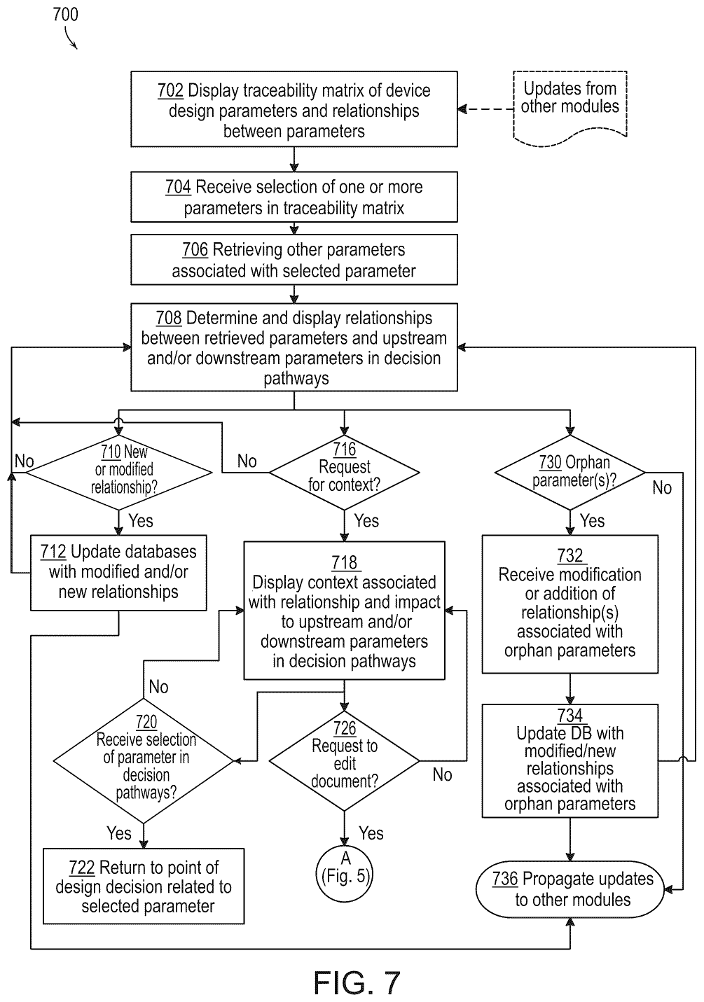

The traceability module 208 is configured to automatically update one or more databases to dynamically integrate updates from one or more design stages 104. The traceability module 208 may be invoked at each point of decision and/or document requirement. A “point of decision”, as used herein, refers to a point in a design stage where a decision regarding one or more device design parameters are made by a user. The point of decision also may be referred to as a point where a design decision is made. The traceability module 208 is described further below with reference to FIG. 7.

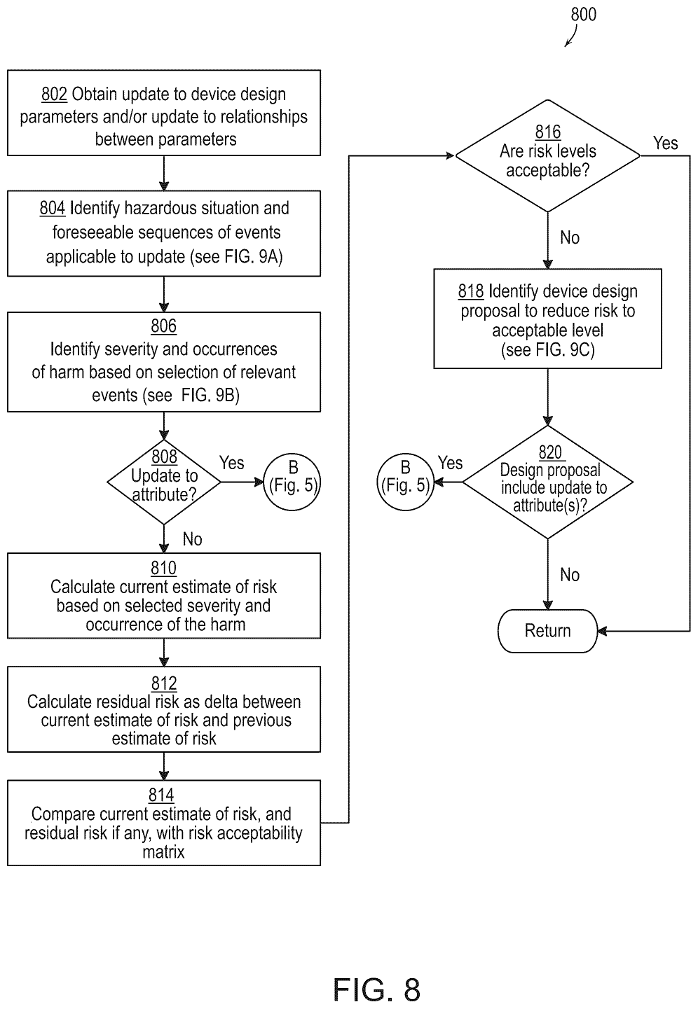

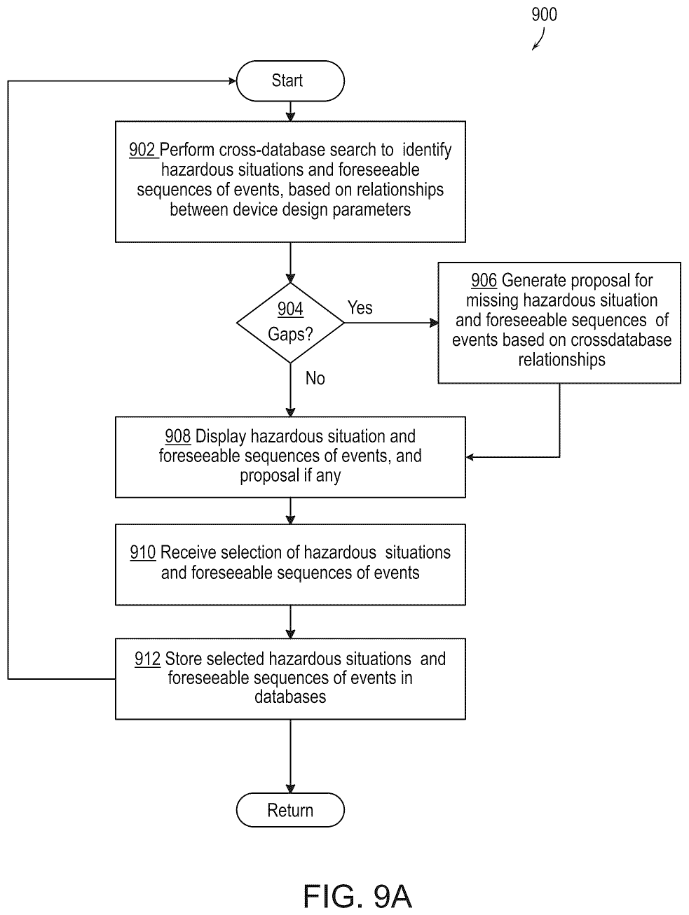

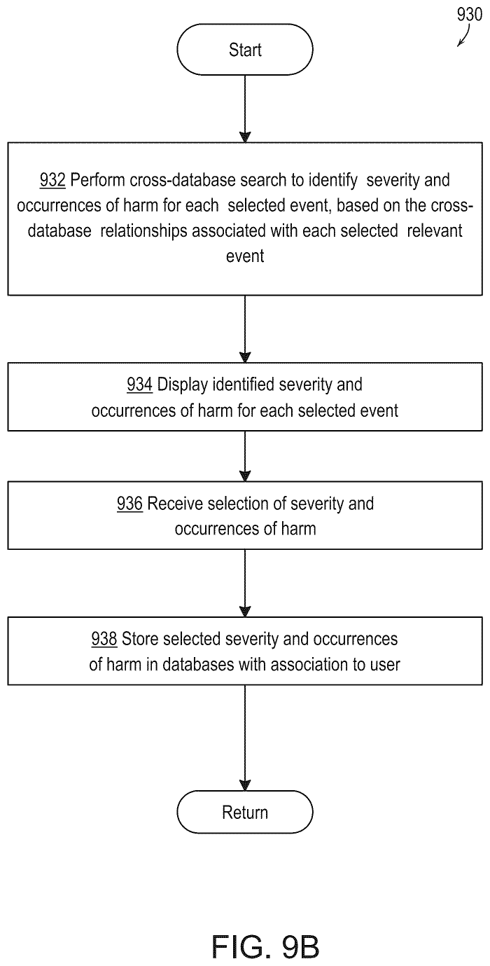

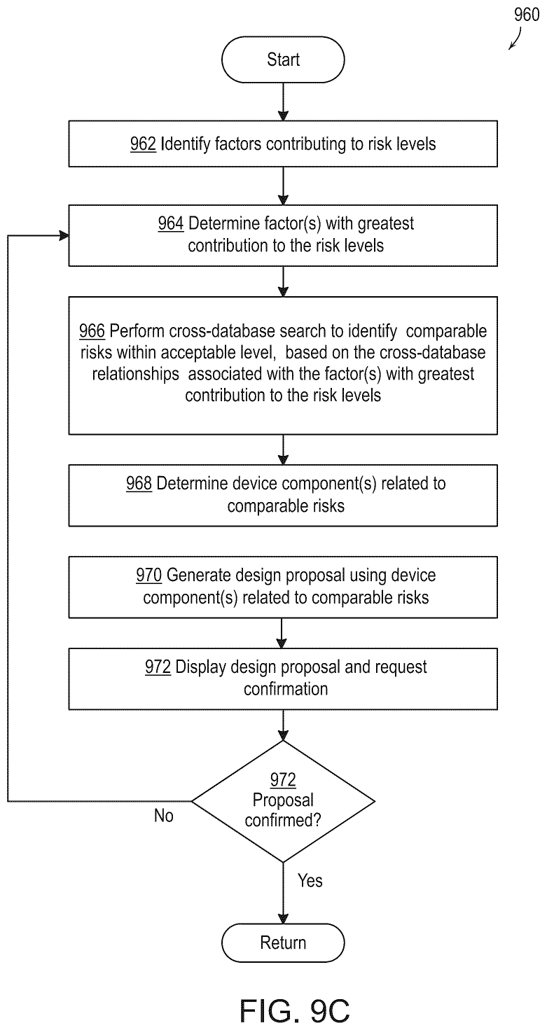

The risk management module 210 is configured to analyze hazardous situations and foreseeable sequence of events, assess the severity and occurrences of harm, and assess comparable risks at one or more design stages 104. The risk management module 210 is described further below with reference to FIG. 8 and FIGS. 9A-9C.

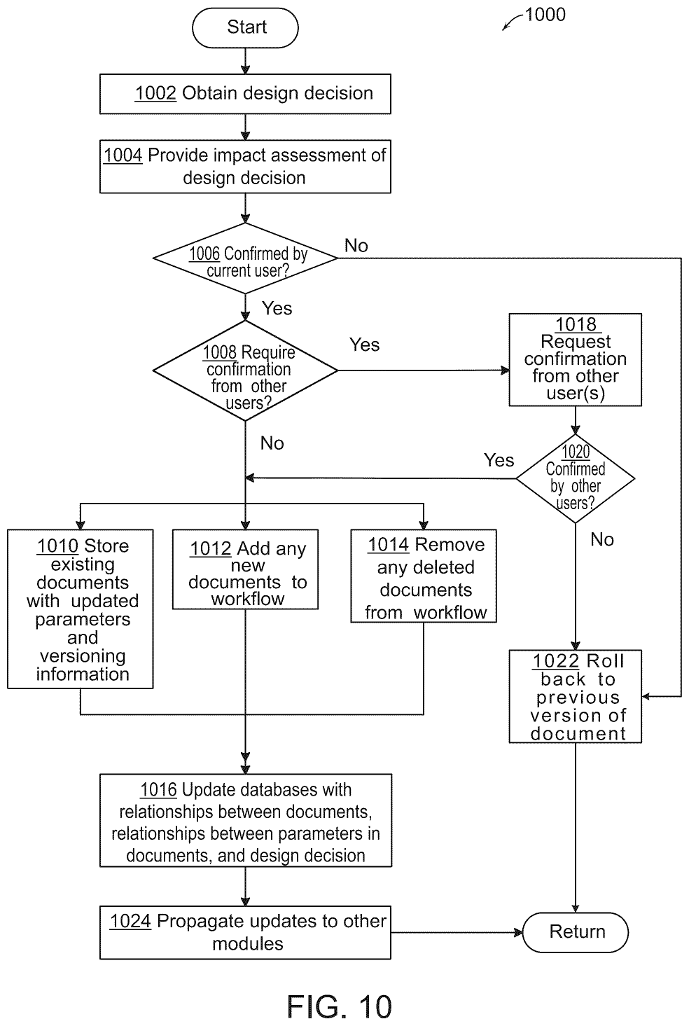

The impact assessment module 212 is configured to provide and document impact assessments. The impact assessment module 212 may be invoked when design decisions are made during the design stages 104. The impact assessment module 212 is described further below with reference to FIG. 10.

The GenAI module 214 may include one or more machine learning models, large language models (LLMs), or other model types configured to interpret regulations and extract requirements for each category: including device structure; design requirement; specification; test cases; and labeling documentation. The GenAI module 214 may be configured to analyze hazardous situations linked with associated device components and/or design characteristics, which aids in the interpretation and input of potential risks. Based on the components, subsystems, and interfaces of a medical device, the GenAI module 214 may be configured to suggest potential hazardous situations across various departments, such as hardware/software engineering, quality, and usability engineering. The GenAI module 214 may further be configured to identify and classify requirements from the standards and regulations. For example, the GenAI module 214 may reference structured repositories such as knowledge graphs, vectorized representations, or other data models, constructed from regulatory and standard documents. Using these sources, the GenAI module 214 may categorize the requirements into component structure, design inputs and outputs, design specifications, verification and validation testing requirements, and document requirements, Each identified requirement may be linked to one or more parameters, metadata, or document templates associated with the corresponding compliance element. The GenAI module 214 may continuously refine its interpretations based on user feedback or system-level validations, thereby improving its capability to provide context-aware, regulation-aligned recommendations over time.

Embodiments of the platform 102 provides an end-to-end workflow with upstream and downstream documents for the design and development of devices or products. The platform 102 automatically propagates updates to the device design parameters from any design stage to the affected upstream and downstream documents, as well as to related processes, such as risk management and impact assessment. The platform 102 may also propagate changes in the external databases to the design stages and processes. The platform 102 stores the updates and their effects in a traceable manner. The updates to the device design parameters may include, for example, changes in device attribute, device profile, entity, regulations, or standards. The platform 102 thus improves the technical field of device design and development, and more specifically to the design and development of medical devices, to ensure compliance with regulatory requirements and industry standards.

Embodiments of the modules 202-214 of the platform 102 are described below in the context of medical device development and compliance for exemplary purposes and are not limiting. The embodiments of the modules 202-214 may also be applicable to other product development and compliance.

### Automated Research Module 202

At the beginning of medical device development, when only an ideation or concept is in place, research across multiple databases may be required, for example, involving: regulatory databases that include product classification, product code, regulations; engineering databases that include technical specifications, test certification, and design standards; marketing databases that include competitive products and company structure; and quality databases that include recalls and adverse events. As changes to the concept occurs, the research must be repeated or updated. As regulation updates are identified, the research process must be repeated, and corresponding documentations must also be updated to comply with the updates regulations. These updates can occur during the middle or late stages of design and development, which may lead to the need to rework existing documentation or add new documentation.

With the platform 102, the research process may be automated by establishing relationships within and across various isolated databases. In response to user inputs, the automated research module 202 presents curated options for the user to choose from and provides intelligent suggestions based on their selections, leveraging Structure Query Language (SQL), knowledge graphs, and machine learning algorithms to refine and optimize search results. As users enter and combine different keywords, the research module 202 automatically updates results from each category (e.g., device structure; design requirement; specification; test cases; and labeling documentation) in real time. This dynamic interaction allows for rapid iteration and exploration of various scenarios without the need for repetitive manual searches. The research module 202 maintains a comprehensive history of search scenarios, which allow reference to previous searches at any time. This feature ensures continuity and aids in tracking the evolution of concepts and ideas. By centralizing the research process, the platform 102 significantly reduces the time required for initial ideation research. By providing scenario history tracking, the platform 102 also reduces the time required for cross-functional alignment. With the platform 102, when a relevant update leads to a change in the databases, the platform 102 undergoes an internal verification process in the background. If a new design decision regarding the device development is required, the platform 102 sends a notification to the user(s) associated with specific new decision.

FIG. 4 includes a flowchart of a method implemented by the automated research module (“research module”) 202 according to some embodiments. The flow 400 is an example flow and not limiting. The flow 400 may be altered, e.g., by having one or more blocks added, removed, rearranged, combined, performed concurrently, and/or having one or more blocks split into multiple blocks.

At block 402, the research module 202 receives a query pertaining to a specific context. Examples of a context may include: product management scope; risk management; hazardous situation; or comparable harm. For example, the research module 202 may receive a query that includes a user-provided keyword, such as a product code, a contract number, etc.

At block 404, the research module 202 performs a cross-database search on databases relevant to the search expressions in the query and cross-database relationships. For example, the research module 202 may determine other entities (devices, standards, testing requirements, etc.) may be related to the keyword based on relationships in a knowledge graph with axis linked to the product characteristics (e.g., a profile of a potential competitive company).

At block 406, the research module 202 displays the search results. The research module 202 may consider the device design parameters or characteristics in determining the most relevant search results using the relationships stored in the databases 216. The automated research module 202 may invoke the Gen AI module 214 to provide smart suggestions regarding the most relevant search results. The platform 202 may present a mechanism for the user to confirm one or more of the suggestions and use the user feedback to revise the search results. For example, the search results may be presented to the user in various ways, such as a knowledge graph. For example, the cross-database relationships may be used to generate a knowledge graph, where the graph may include an axis that is linked to the product characteristics of the device, a profile of a potential competitive company, or some other relationship based on the query.

At block 480, the research module 202 receives a selection of relevant results from the user. For example, the research module 202 may dynamically update the knowledge graph based on the user's selection.

At block 410, the research module 202 stores the query, the search results, and the selected results, and associates them with the user in a scenario history database 220.

In parallel with blocks 402-410, the research module 202 performs blocks 414-426. At block 414, the research module 202 monitors external databases changes (e.g., changes to the FDA regulations database 232 and/or the ISO standards database 234).

At block 416, the research module 202 determines whether one or more changes are detected. If so, at stage 418, the research module 202 determines the impact of the change to one or more attributes stored in the databases 216.

At block 420, the research module 202 determines whether the impacted attribute(s) are relevant, i.e., applicable to the device under development. If the impacted attribute(s) are relevant, then at block 422, the research module 202 sends a notification of the impacts to the user associated with the attribute based on the responsibilities assigned to the user's role. If the research module 202 receives, at block 424, an acceptance or confirmation of the impacts from the user, then at block 426, the research module 202 modifies the device design attributes for the medical device according to the accepted change and stores the change in the databases 216. The modified device design attributes may then be retrieved and applied during the various design stages. In this way, changes to attributes due to changes in regulations or standards may be propagated to the various design stages.

### Automatic Cross-Functional Alignment Module 204

Based at least on the device design attributes, the automatic cross-functional alignment module 204 (“alignment module”) provides a dynamic, real-time product development workflow with a graphical interface through which a user may interact. For example, the alignment module 204 may present a graphical workflow showing documents corresponding to the requirements according to regulations or standards applicable to the device under development, the tasks required to complete each document, and the document's relationships to other documents (i.e., the upstream and downstream documents). The specific documents in the graphical workflow may be based on the role assigned to the user, where documents outside of the user's role is not included in the graphical workflow. The user may select any of the documents in the graphical workflow to perform the required tasks. During the performance of a task, the user may make design decisions, such as making an update to a device attribute value. The alignment module 204 may then propagate the updated device attribute value to upstream and downstream documents containing the same device attribute and/or a dependent device attribute. The graphical workflow may further be configured to enforce a required order of completion for the documents. The alignment module 204 processes the interactions with the user and facilitates cross-functional alignments, including dynamically updating relationships and device attributes based on the user interactions. For example, once a document is complete, the alignment module 204 automatically updates upstream and downstream documents based on the completed document, where an updated device attribute value may be propagated to upstream and downstream documents containing the same or dependent device attribute. The updates to the device attributes may also be propagated to one or more of the other modules in the platform 102 and/or stored in the databases 216. The design decision by the current user may impact one or more documents in other user(s) workflow(s), and the alignment module 204 may update the graphical workflow(s) of the other user(s). Similarly, design decisions by other user(s) may impact one or more documents in the current user's workflow, and the alignment module 204 may update the graphical workflow of the current user. The alignment module 204 thus automatically propagates a design decision throughout the overall device development workflow and facilitates automatic alignment across regulatory, product management, engineering, quality, and other cross-functional teams due to changes to attribute values.

FIG. 5 includes a flowchart of a method implemented by the automatic cross-functional alignment module (“alignment module”) 204 according to some embodiments. The flow 500 is an example flow and not limiting. The flow 500 may be altered, e.g., by having one or more blocks added, removed, rearranged, combined, performed concurrently, and/or having one or more blocks split into multiple blocks.

At block 502, the alignment module 204 displays a device development portal, including documents, where each document is associated with the one or more device design attributes of the device under development and may be an output for a regulatory submission. Each document may include one or more tasks, queries, or processes to be performed by a user in order to complete the document. Optionally, the alignment module 204 may display the tasks, queries, or processes as a selectable item on the interface. In an example embodiment, each device under development is associated with a workflow or “project”. Each user associated with the project may be assigned a role based on the functional team to which the user is assigned and/or the user's responsibilities within the assigned team. For example, the user's role may be associated with regulatory, engineering, marketing, and/or quality processes, and the alignment module 204 may customize the product development portal for the user to view and complete tasks associated with one or more of these processes. The product development portal may further be customized to incorporate the company SOP and existing documents 230. This customization of the portal ensures that users have access to the most relevant information and tools needed to perform their specific tasks.

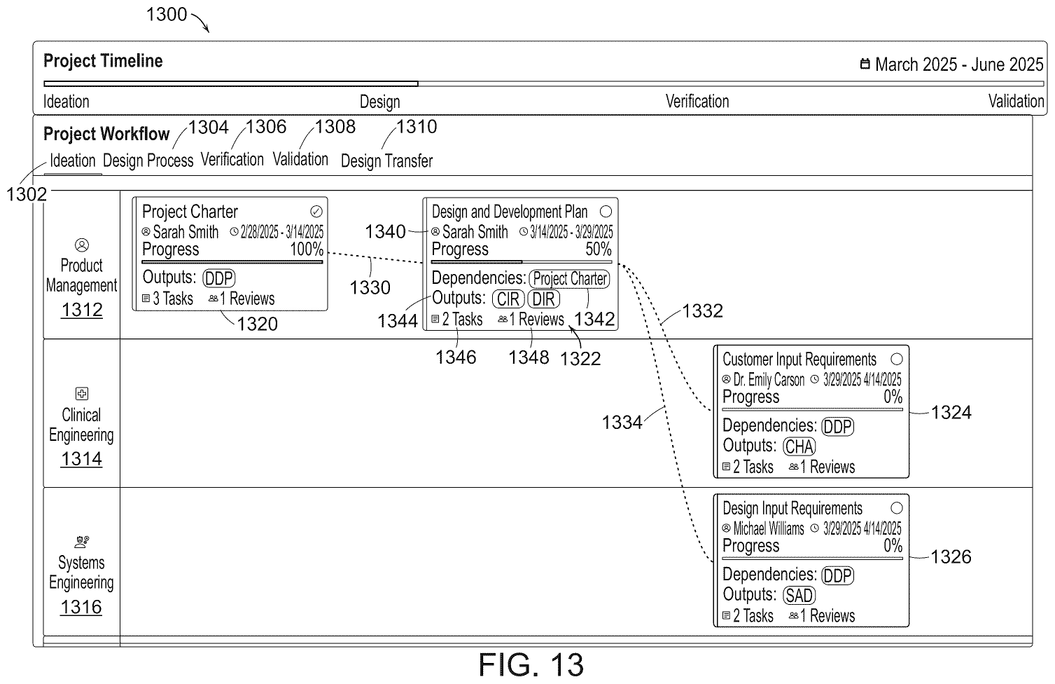

FIG. 13 includes an example device development interface of an alignment module. The interface 1300 may display selectable “tabs” representing the design stages or processes, such as Ideation 1302, Design Process 1304, Verification 1306, Validation 1308, and Design Transfer 1310. The portal 1300 may further include documents 1320-1326 and teams or departments 1312-1316 displayed as horizontal “bands”. The documents 1320-1326 may be displayed in the bands 1312-1316 associated with the team that is responsible for the document. Connections, via lines 1330-1334, indicate relationships between the documents.

For example, as illustrated in FIG. 13, the ideation tab 1302 has been selected. Under the ideation tab 1302, The teams include product management 1312, clinical engineering 1314, and systems engineering 1316, and others (not shown). The project charter document 1320 and the design and development plan document 1322 are displayed in the product management band 1312 to indicate that the product management team is responsible the documents 1320, 1322. The customer input requirements document 1324 is displayed in the clinical engineering band 1314 to indicate that the clinical engineering team is responsible for this document 1324. The design input requirements document 1326 is displayed in the systems engineering band 1316 to indicate that the systems engineering team is responsible for this document 1326. The connection 1330 indicates a relationship between the project charter document 1320 and the design and development plan 1322. The connections 1332 and 1334 indicate relationships between the design and development plan document 1322 and the customer input requirements document 1324 and the design input requirements document 1326. The order of display for the document 1320-1326, in conjunction with the connections 1330-1334, may be used to indicate an order of flow or dependencies between the documents 1320-1326.

Optionally, a display of a document in the workflow may include information related to the document. For example, the design and development plan document 1322 may include the user assigned to the document 1340 (e.g., Sarah Smith), the dependencies 1342, i.e., the document(s) on which the document 1322 depends (e.g., Project Charter), the outputs 1344 of the design and document 1322 (e.g., CIR and DIR), a selectable object 1346 for the tasks associated with the document 1322, and a selectable object 1348 for the required reviews of the document 1322 prior to completion.

Returning to FIG. 5, at block 504, the alignment module 204 receives a selection of a current document from the user.

At block 506, in response to the selection, the alignment module 204 displays the contents of the current document, which may include attributes, and one or more tasks to be performed to complete the document. The attributes in the document may include one or more parameters, each requiring values.

At block 508, the alignment module 204 receives, from the user, a selection of a current attribute in the current document.

At block 510, in response to the selection of the current attribute, the alignment module 204 determines whether a proposed update to the current attribute in the current document is received, i.e., receive a new value or a changed value for the current attribute.

At block 512, the alignment module 204 requests that the user confirm the proposed update to the current attribute. If the update is not confirmed, then the alignment module 204 discards the proposed update and returns to block 506.

At block 514, in response to receiving a confirmation of the update, the alignment module 204 updates the current attribute in the current document with the new or changed value.

At block 516, the alignment module 204 updates attributes in downstream and upstream documents affected by the updated current attribute in the current document. The alignment module 204 further updates the attributes stored in the device profiles database 222. For example, the updating of the current attribute in the current document may automatically trigger the invocation of a query for other attributes affected by the update, based on the established relationships within and across databases, as stored in the cross-database relationships database 218. For example, an attribute may be affected when or another document in the workflow also includes the updated attribute or includes another attribute whose value depends upon the value of the updated attribute. The relationships may include requirements, which define relationships that are optional and/or mandatory for the attribute. “Mandatory” requirements include requirements set by FDA regulations 232 and ISO standards 234. The alignment module 204 may automatically, dynamically and in real-time, update the attribute in the downstream and/or upstream documents affected by the updated current attribute in the current document. Optionally, the alignment module 204 may display the affected attributes from the results of the query to the user, and/or display the downstream or upstream documents impacted by the update, and request confirmation of the update of the current attribute in the current document prior to applying the update to the downstream and/or upstream documents. Optionally, alignment module 204 may receive a selection of an impacted downstream or upstream document and return to block 506 to display the selected downstream or upstream document as the current document. If the user fails to confirm the update to the current attribute, the alignment module 204 may reverse or rollback the attribute update throughout the workflow. The alignment module 204 may further trigger a request for suggestions from the GenAI module 214, where the suggestions may include correlated relationships that are optional and based on historical data and patterns. The alignment module 204 may display the suggestions and request that the user confirm whether the suggestions are applicable to the current document. The user's response may then be provided as an input to the GenAI module 214 to improve the GenAI module's accuracy. This adaptive learning enhances the platform's accuracy over time, ensuring increasingly relevant and valuable insights for users. The affected downstream and/or upstream documents may be assigned to the user or to another user. The affected downstream and/or upstream documents may be assigned to the same team as the user or to a different team. Similarly, an attribute may be updated by another user, and the alignment module 204 may determine that the one or more documents in the user's workflow are affected by the update of the attribute. The alignment module 204 then updates the user's workflow accordingly, and the user may be notified of the updates by the other user.

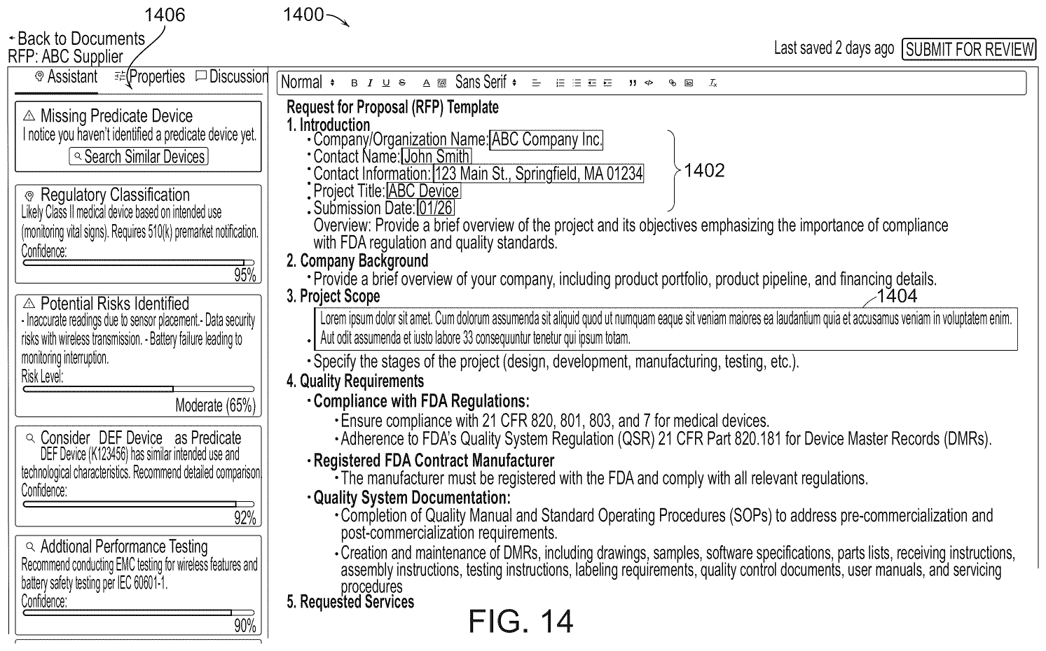

For example, as illustrated in FIG. 14, in block 504, the alignment module 204 may receive a selection of a request for proposal (RFP). In block 506, the alignment module 204 displays the contents of an RFP document 1400 according to an RFP template. The RFP document 1400 includes one or more device design attributes 1402-1404 associated with the RFP document 1400. For example, the device design attributes may include the introductory attributes 1402 (e.g., company/organization name, contact name, contact information, project title, and submission date) and the project scope attribute 1404. In block 508, the alignment module 204 may receive a selection of the project scope attribute 1404, and in block 510, receive a proposed new or changed value for the project scope. Assuming that the updated project scope is confirmed in block 512, the alignment module 204 updates the project scope in the RFP document 1400 in block 514. In block 516, the alignment module 204 also updates any documents downstream or upstream from the RFP document 1400 in the workflow affected by the update in the project scope. Suggestions 1406 from the GenAI module 214 may be displayed with the RFP document 1400.

Returning to FIG. 5, at block 518, the alignment module 204 determines whether any other documents are to be added and/or deleted due to the update to the current attribute.

At block 520, if there are other documents to be added and/or deleted, then the alignment module 204, at block 526, updates the workflow to add and/or delete the other documents and returns to block 502.

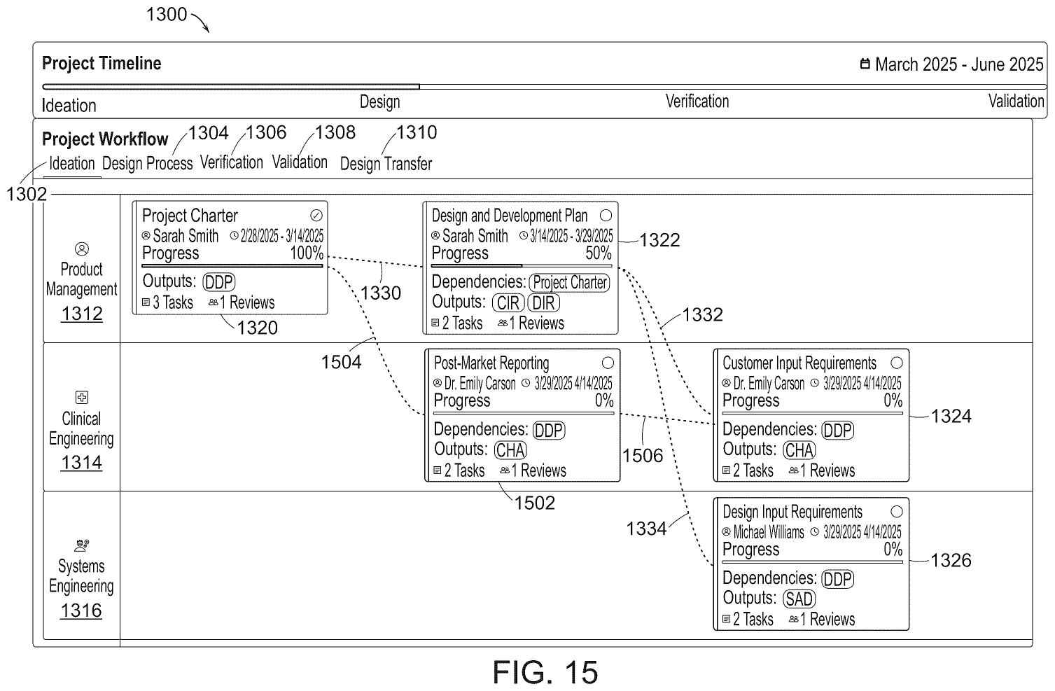

For example, as illustrated in FIG. 15 and continuing the example of FIG. 14, the alignment module 204 determines that a post-market reporting document is to be added due to the update to the current attribute. The alignment module 204 updates the workflow to add the post-market reporting document 1502. The alignment module 204 further determines that the post-market reporting document 1502 is assigned to the clinical engineering team 1314 and has connections with the project charter document 1320 and the customer input requirements document 1324. The alignment module 204 thus displays the post-market reporting document 1502 in the clinical engineering band 1314 and with connections 1504 to the project charter document 1320 and connection 1506 with the customer input requirements document 1324.

At block 522, if there are no other documents to be added and/or deleted, then the alignment module 204 determines whether the current document has been completed. If the current document has not been completed, then the alignment module 204 returns to block 508, where the alignment module 204 may receive a selection of another attribute in the current document.

At block 524, if the current document has been completed, then the alignment module 204 requests approval of the completed current document from the user. Once the current document is completed and approved, the alignment module 204 returns to block 502. The user may then select a different document in the workflow.

The alignment module 204 tracks each design decision (e.g., an update to an attribute) by the user and associates the decision with the user's role, as well as with cross-functional tasks. The workflow for the device under development may be automatically updated with additional documents, modified documents, and/or deleted documents as design decisions are made by multiple users. For example, when a design decision is made, the alignment module 204 may display the time stamp for the design decision and the identity of the user in another user's workflow. The workflow may further provide an option to view the audit trail of the decision path, along with document versioning information, for a user to understand context of the design decision.

### Smart Regulation Interpretation Module 206

The device development workflow may depend on the regulations related to the device. The regulations are often complex, requiring the identification of applicable regulations across multiple databases, for product concepts and refining decisions through the research and development period. The regulations may encompass design, test, labeling, usability and/or other requirements. Changes to the regulations may lead to the need to identify gaps in parameters, such as the attributes, profiles, and document requirements for the device, which may in turn lead to changes in the device development workflow. Similarly, decision changes may lead to new applicable regulations, which would require the propagation of changes throughout the device development workflow. The smart regulation interpretation module (“interpretation module”) 206 automatically monitors external databases for regulation changes, such as changes to the regulations in the FDA regulations database 220, and dynamically integrates the changes in the databases 216, as well as the device development workflow, which in turn dynamically updates the workflows for the users.

FIG. 6 includes a flowchart of a method for monitoring and integrating regulation changes according to some embodiments. The flow 600 is an example flow and not limiting. The flow 600 may be altered, e.g., by having one or more blocks added, removed, rearranged, combined, performed concurrently, and/or having one or more blocks split into multiple blocks.

At block 602, the interpretation module 206 receives one or more regulations from an external database, which may include the FDA regulations database 220 and/or the ISO standards database 222, as well as other databases. For example, the interpretation module 206 may receive changes in the regulations from a periodic monitoring of the regulations in the FDA regulations database 232.

At block 604, the interpretation module 206 extracts information about device design parameters from the one or more regulations. For example, the interpretation module 206 may extract device attributes, profiles, and/or document requirements. The interpretation module 206 may invoke the GenAI module 214 to extract requirements for device structure, design requirements, specification, test cases, labeling, documents, and/or other categories of parameters.

At block 606, the interpretation module 206 determines one or more proposed relationships between the parameters based on the extracted information.

At block 608, the interpretation module 206 automatically optimizes the one or more proposed relationships. For example, the GenAI module 214 may analyze the extracted data and generate suggestions for additional or refined relationships that are not initially included in the proposed set. These suggestions may be based on semantic similarity, contextual co-occurrence, or known hierarchical structures within the databases. The optimized relationships may then be re-ranked or weighted according to confidence levels inferred from prior validated mappings. The resulting set of relationships may be presented to the user in various formats, for instance, as a dynamically updated graph highlighting new connections or as a ranked list of candidate relationships for user confirmation. Through iterative validation, the module 206 may continuously improve its accuracy and relevance in identifying latent or implicit relationships among entities.

At block 610, to ensure accuracy, the interpretation module 206 displays the proposed relationships to a user and requests verification. For example, the interpretation module 206 may display the proposed relationships to a user via the workflow. The user may be assigned a role that corresponds to the proposed relationships.

If the interpretation module 206 receives verification at block 612, then the interpretation module 206, at block 614, stores the verified device design parameters and the relationships to the cross-database relationships database 214. If confirmation is not received from the user, the proposed relationships are discarded.

In parallel with blocks 602-614, the interpretation module 206 performs blocks 616-628 to monitor the regulations in the external databases (e.g., FDA regulations database 232 and/or the ISO standards database 234) and to ensure that the relationships between the parameters are updated when changes occur.

At block 616, the interpretation module 206 monitors external databases (e.g., FDA regulations database 232 and ISO standards database 234) for changes in the regulations. For example, the interpretation module 206 may monitor the external databases periodically at configured time intervals.

At block 618, the interpretation module 206 determines whether one or more changes are detected. If so, at block 620, the interpretation module 206 performs blocks 602-614, as described above. The interpretation module 206 further determines the impact of the change in the regulations to the device design parameters stored in the databases 216.

At block 622, the interpretation module 206 determines whether the impact of the change of the regulations includes impacts to the current device design. If so, at block 624, the interpretation module 206 sends a notification of the impacts to the current device design to a user of the platform 102. If the user, at block 626, accepts or confirms the impacts to the current device design, then at block 628, the interpretation module 206 modifies the current device design and associated workflow and propagates the modification to the other modules of the platform 102.

### Automatic End-to-End Traceability Module 208

Traceability is critical in ensuring every aspect of the medical device's development, from design to production, meets safety and efficacy standards. The automatic end-to-end traceability module (“traceability module”) 208 enables an export of an overview of all traceability as part of a regulatory submission, such as an FDA submission. For example, the export may be part of the DHF 312 or part of the documentation of processes 306. The traceability module 208 may also import or sync with commercially available requirement management tools such as JIRA, JAMA, etc., where the traceability module 208 exports and compiles information from various tools to create the final documentation for submission. The traceability module 208 automates the updating of associated input and output requirements as design changes occur and automatically updates and records decision paths for traceability. Inefficiencies and errors that otherwise may occur due to the fragmentation of processes, manual identifications, manual recreation of decision paths, manual compilation of the final submission documentation, and use of disparate tools may be avoided or significantly reduced. When design changes occur, alignment bottlenecks, reliance on meetings, and the reliance on the collective experience, qualifications, and memory of the users may be decreased or avoided.

FIG. 7 includes a flowchart of a method for automatic end-to-end traceability according to some embodiments. The flow 700 is an example flow and not limiting. The flow 700 may be altered, e.g., by having one or more blocks added, removed, rearranged, combined, performed concurrently, and/or having one or more blocks split into multiple blocks.

At block 702, the traceability module 208 displays a traceability matrix of device design parameters and relationships between the parameters. The traceability matrix may include updates to device design parameters from other modules of the platform 102. For example, the traceability module 208 may display the traceability matrix in response to the receipt of a user request. The traceability matrix provides an end-to-end traceability visualization, where each parameter is shown in the matrix with connections to other parameters, such as system requirements, subsystem requirements, technical specifications, test cases, and/or test results. For example, the traceability module 208 may build the traceability matrix, where each device component has at least one system requirement, each system requirement has at least one subsystem requirement, each subsystem requirement has at least one technical specification, each technical specification has at least one test case, and each test case has at least one test result. The traceability matrix represents a current state of the device under development based on the requirements from regulations and ISO standards and the relationships between the requirements. The traceability matrix incorporates the design decisions received via one or more of the modules in the platform 102.

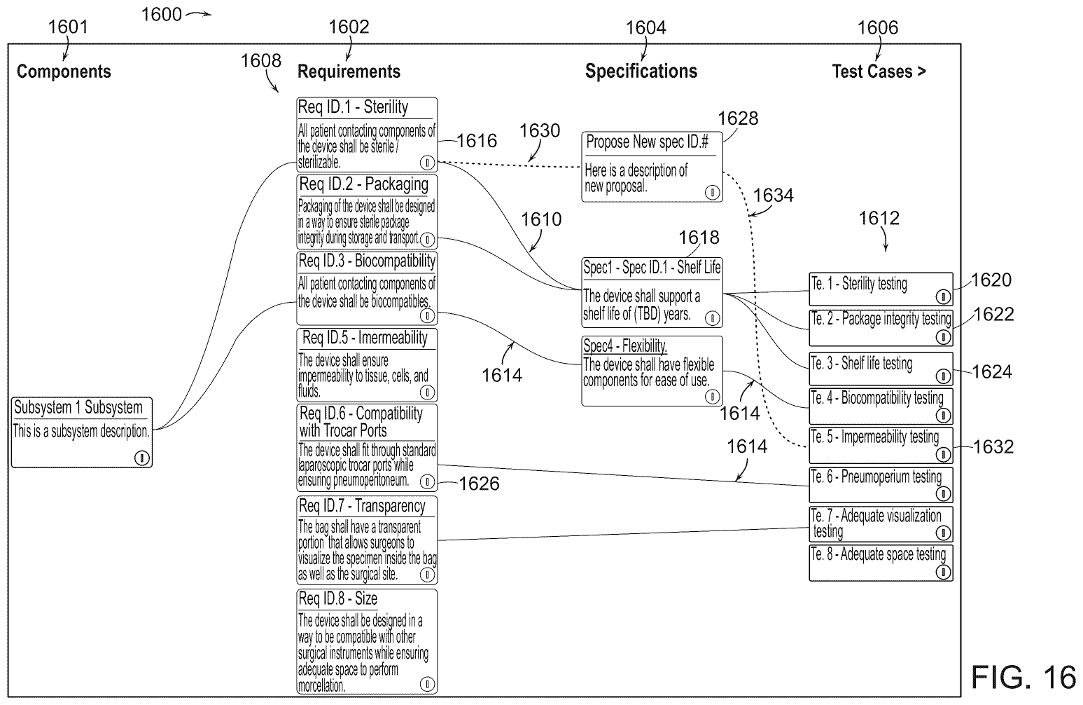

FIG. 16 includes an example interface of a traceability matrix. The example traceability matrix interface 1600 displays the parameters, such as components 1601, requirements 1602, specifications 1604, test cases 1606, and test results (not shown), with connections via lines 1614 to one or more values of the parameters 1608, 1610, and 1612. For example, the requirement for sterility 1616 may be that “all patient contacting components of the device shall be sterile/sterilizable”. The sterility requirement 1616 is related to the specification with a shelf life 1618. The shelf life 1618 is related to sterility testing 1620, package integrity testing 1622, and shelf life testing 1624. Optionally, the interface 1600 may include a selectable object 1626 with each parameter value to request additional information regarding the parameter value, such as identifying the document(s) related to the parameter value.

Returning to FIG. 7, at block 704, the traceability module 208 receives a selection of one or more elements in the traceability matrix.

At block 706, the traceability module 208 retrieves one or more other parameters associated with the selected parameter.

At block 708, the traceability module 208 determines and displays the relationships between the retrieved parameters and upstream and/or downstream parameters in decision pathways. For example, two parameters with a relationship may be shown with a connecting line, where each series of connected parameters represent a decision pathway.

Upon determining the relationships between the retrieved parameters and the upstream and/or downstream parameters, the traceability module 208 performs three parallel sets of blocks. In a first set of blocks 710-712, the traceability module 208 updates the databases 216 with any new or modified relationships. In a second set of blocks 716-728, the traceability module 208 updates the upstream and/or downstream parameters in the decision pathways. In a third set of blocks 730-734, the traceability module 208 updates the databases 216 with relationship(s) associated with orphan elements.

Regarding the first set of blocks, at block 710, the traceability module 208 determines whether the relationships between the retrieved parameters and the upstream and/or downstream parameters include any new or modified relationships. For example, new or modified relationships may be required due to changes in the device design.

At block 712, if the relationships include new or modified relationships, then the traceability module 208 updates the databases 216 with the modified and/or new relationships. The traceability module 208 then returns to block 708.

At block 736, the updates, i.e., the new and modified relationships, are propagated to the other modules in the platform 102.

Regarding the second set of blocks, at block 716, the traceability module 208 determines whether a request for a context associated with the relationships is received. A context, as used herein, refers to the historical design decision(s) that has led to the current state of the parameters. By requesting the context, the traceability module 208 determines the upstream and/or downstream parameters and documents in the decision pathway applicable to the historical design decisions.

At block 718, in response to a request for context, the traceability module 208 displays the context associated with the relationships and impact to the upstream and/or downstream parameters in the decision pathways. For example, the decision pathway may be displayed with a plurality of parameters connected by lines. If no connecting lines exist for a parameter, the traceability module 208 may prompt the user to add a relationship or select from one or more suggested relationships. Referring again to FIG. 16, for example, an established relationship between the sterility parameter 1616 and the shelf life parameter 1618 may be displayed as a solid line 1610, while a suggested relationship between the sterility parameter 1616 and the propose new spec parameter 1628 may be displayed as a dotted line 1630. Similarly, a suggested relationship between the propose new spec parameter 1628 and the impermeability testing parameter 1632 may be displayed as a dotted line 1634. When a connection is made or modified, the traceability module 208 determines what user roles are associated with the newly connected parameters and sends a notification to the users associated with the roles. For example, the traceability module 208 may display the relationships as part of the device development workflow. For example, the traceability module 208 may display a context bubble in the device development workflow that shows the decision pathway.

At block 720, the traceability module 208 may receive a selection of a parameter in the decision pathways. At block 722, in response to a selection of a parameter in a decision pathway, the traceability module 208 returns to the point of decision related to the selected parameter. A user may thus navigate from the traceability matrix directly to the module with the point of decision.

At block 726, the traceability module 208 may also receive a request to edit a downstream document. In response to a request to edit a downstream document, the traceability module 208 returns to block 506 of FIG. 5. A user may thus navigate from the traceability matrix to a document in the workflow.

Regarding the third set of blocks, at block 730, the traceability module 208 may identify and one or more orphan parameters. Each parameter should have at least one link to another parameter. An orphan parameter, as used herein, refers to a parameter with no links to another parameter.

At block 732, the traceability module 208 receives one or more modifications or additions of relationships associated with the orphan parameters. In some embodiments, the traceability module 208 may invoke the GenAI module 214 to assist in contextual interpretation of the orphan parameters and to generate recommendations for potential indirect or inferred relationships based on the modified or newly added relationships. The GenAI module 214 may analyze the underlying data context, historical mappings, and semantic patterns across prior projects or related device profiles to identify parameters that exhibit logical or functional correlation. These correlations may include inferred dependencies, design intent alignments, or regulatory linkages that are not explicitly captured within the relational schema. The traceability module 208 may further invoke the GenAI module 214 to generate recommendations for indirect relationships for the orphan parameters based on the modified or additional relationships. The traceability module 208 may prompt the user to confirm the suggestions generated by the GenAI module 214. User feedback, including confirmations or rejections of suggestions, may be transmitted back to the GenAI module 214 to refine its subsequent recommendations and enhance adaptive accuracy. if confirmed, includes the confirmed indirect relationships to the modified and/or new relationships.

At block 734, the traceability module 208 updates the databases 216 with the modified and/or new relationships associated with the orphan parameters. Such updates may also include metadata linking the source and rationale of inferred relationships, enabling downstream traceability and auditability of GenAI-assisted decisions.

At block 736, the updates, i.e., the modified and/or new relationships associated with the orphan parameters, are propagated to the other modules in the platform 102.

### Risk Management Module 210

Risk management is a critical process requirement for any medical device, spanning multiple design stages. However, it is often implemented as an afterthought, leading to significant challenges and inefficiencies, primarily in the form of design and document rework. Further, there are many design standards specifically related to medical devices, each addressing different aspects of the development, manufacturing, and management processes. These standards are constantly updated by multiple agencies and varying by country, posing significant challenges for cross-functional teams. Navigating these standards poses significant challenges for cross-functional teams.

To address these challenges, the risk management module 210 integrates with databases containing historical events, recalls, and other risk-related data, databases of various regulatory agencies, and industry best practices. The risk management module 210 may query and provide similar or suggestions of design standards applicable to the device under development. The risk management module 210 automates the retrieval and analysis of historical risks, significantly reducing manual labor and the potential for oversight. The risk management module 210 also continuously monitors and updates databases with the modified standards and regulations from multiple sources and countries and sends notifications to users to confirm the applicability of the modified standards and regulations to the design of the device under development.

The risk management module 210 may further extract, from other databases, hazardous situations linked with associated components and design characteristics of the device under development. This aids in the interpretation and input of the potential risks, minimizing manual error and ensuring consistency.

The risk management module 210 may invoke the GenAI module 214 to suggest potential hazardous situations related to the components of the device under development, including hardware and/or software engineering, quality, and usability engineering. Based on user feedback regarding the suggestions, the risk management module 210 may learn and adapt accordingly. For example, the risk management module 210 may utilize generative AI and Retrieval Augmented Generation (RAG) to extract requirements and standards and to generate a knowledge graph and/or vector databases. Each identified requirement may have associated parameters or document templates. Requirements may be categorized into component structure, design requirement, design specifications, testing requirement and document requirements utilizing the knowledge graph and/or vector databases. Standards may be mapped to specific device components and development processes. When a new version of the standard is published, the risk management module 210 may perform a gap analysis to identify any gaps in the hazardous situations and foreseeable sequences of events and revise the workflow accordingly.

The risk management module 210 creates a centralized knowledge base, storing interpreted standards and their implications for various device components. This allows for the access and sharing of project information across teams, ensuring transparency, and reducing silos. External subject matter expert consultants can also be provided access to the platform 102, where decisions made by users may be displayed for consultants for review. The risk management module 210 aids in impact assessment, where previous design decisions and/or downstream documentation may be revisited, and, if any new design decision is made, the risk management module 210 automatically updates the associated parameters based on the new design decision and any impacted documents.

The risk management module 210 includes a user interface through which users may request definitions and references across multiple standard databases. For any upstream design changes, the risk management module 210 automatically populates associated hazardous situations in real-time, reducing the potential for human error and ensures that the risk management is continuously updated in line with design changes. The risk management module 210 may be applicable and adaptable across different disciplines.

FIG. 8 includes a flowchart of a method for automatic risk management according to some embodiments. The flow 800 is an example flow and not limiting. The flow 800 may be altered, e.g., by having one or more blocks added, removed, rearranged, combined, performed concurrently, and/or having one or more blocks split into multiple blocks.

At block 802, the risk management module 210 obtains an update to one or more device design parameters and/or an update to one or more relationships between the parameters. The update may be obtained from another module in the platform 102. For example, the update may occur via the research module 202 (see FIG. 4), the alignment module 204 (see FIG. 5), the interpretation module 206 (see FIG. 6), and/or the traceability module (see FIG. 7).

At stage 804, in response to receiving the update, the risk management module 210 identifies hazardous situation and foreseeable sequence of events applicable to the update of the parameter. A “hazardous situation”, as used herein, includes potential scenarios with risks of harm to a patient due to the use of the device under development. A “foreseeable sequence of events”, as used herein, includes a sequence of events that may lead to the hazardous situation. For example, the device may include a cauterizer, where the foreseeable sequence of events may include the user using the cauterizer to cut tissue, and the hazardous situation may include the unintentionally burning of the tissue. For another example, the foreseeable sequence of events may include the device being damaged during use, such as from the device being dropped, and the hazardous situation may include the energized component becoming exposed.

FIG. 9A includes a flowchart of a method for identifying hazardous situation and foreseeable sequence of events, according to some embodiments. The flow 900 may be altered, e.g., by having one or more blocks added, removed, rearranged, combined, performed concurrently, and/or having one or more blocks split into multiple blocks.

At block 902, the risk management module 210 performs a cross-database search, e.g., on the databases 216, to identify hazardous situations and foreseeable sequences of events applicable to the relationships between the device design parameters.

At block 904, the risk management module 210 determines whether any gaps exist in the identified hazardous situations and foreseeable sequences of events, based on regulatory requirements and/or ISO standards. For example, the risk management module 210 may determine that, based on a benchmark or comparison with a predicate device, an expected related downstream document or attribute is missing or has an empty value. For example, an FDA predicate device is a legally marketed medical device that serves as a point of comparison to establish that a new medical device is substantially equivalent to it in terms of safety and effectiveness. If the new medical device is found to be substantially equivalent to its predicate, then the new medical device receives the same risk classification as the predicate. By benchmarking or comparing the medical device with the predicate device, gaps in the hazardous situations and foreseeable sequences of events may be identified.

If one or more gaps are identified, then at block 906, the risk management module 210 generates a proposal for the missing hazardous situations and foreseeable sequences of events. The proposal may be generated by the GenAI module 214.

At block 908, the risk management module 210 displays the hazardous situation and foreseeable sequences of events, and the proposal if any.

At stage 910, the risk management module 210 receives a selection of one or more hazardous situation and foreseeable sequences of events. For example, the risk management module 210 may receive a selection of events a user considers to be applicable to the device under development.

At stage 912, the risk management module 210 stores the selected hazardous situation and foreseeable sequences of events in the databases 216. The events may be stored with an association to the user. The selected hazardous situation and foreseeable sequences of events may be provided as an input to the GenAI module 214 to improve the Gen AI module's accuracy.

Returning to FIG. 8, at block 806, the risk management module 210 identifies one or more severity and occurrences of harm based on the selected hazardous situation and foreseeable sequences of events from block 910 of FIG. 9A. A “severity and occurrences of harm”, as used herein, includes the potential level of harm to a patient due to the hazardous situation. For example, returning to the example with the cauterizer, the harm may include the potential of an electric shock due to an exposed energized component, and the severity may be probabilities of the cauterizer being dropped and the energized component becoming exposed as a result.

FIG. 9B includes a flowchart of a method for identifying severity and occurrences of harm, according to some embodiments. The flow 930 may be altered, e.g., by having one or more blocks added, removed, rearranged, combined, performed concurrently, and/or having one or more blocks split into multiple blocks.

At block 932, the risk management module 210 performs a cross-database search to identify severity and occurrences of harm for each selected event from block 910 of FIG. 9A, based on the cross-database relationships 218 associated with each of the selected events.

At block 934, the risk management module 210 displays the identified severity and occurrences of harm for each selected event.

At block 936, the risk management module 202 receives a selection of one or more of the severity and occurrences of harm.

At block 938, the risk management module 202 stores the selected severity and occurrences of harm in the databases 224. The selected severity and occurrences of harm may be stored with an association to the user. The selected severity and occurrences of harm may be provided as an input to the GenAI module 214 to improve the Gen AI module's accuracy.

Returning to FIG. 8, at block 808, the risk management module 210 determines whether the selected severity and occurrences of harm results in an update to an attribute. If the selected severity and occurrences of harm results in an update to an attribute, then the risk management module 210 returns to block 506 of FIG. 5. Otherwise, the risk management module 210 proceeds to block 810.

At block 810, the risk management module 210 calculates a current estimate of risk based on the selected severity and occurrences of the harm. Various methods of calculating the current estimate of risk may be used. For example, the current estimate of risk may be calculated based on a combination of a probability of the occurrence of the harm and a probability of reducing the likelihood of harm occurring through potential risk control measures.

At block 812, if a previous estimate of risk exists, then the risk management module 210 also calculates a residual risk as a delta between the current estimate of risk and the previous estimate of risk. Optionally, the risk management module 210 may request confirmation from the user of the calculated current estimate of risk and the residual risk before proceeding to block 814.

At block 814, the risk management module 210 compares the current estimate of risk, and the residual risk if any, with a risk acceptability matrix. For example, a risk acceptability matrix may include risk levels for different combinations of probabilities of occurrences of harm and severities of harm. The current estimate of risk may match a certain combination or a range of combinations.

At block 816, the risk management module 210 determines whether the risk levels are acceptable based on the comparison between the current estimate of risk and the residual risk (if any) and the risk acceptability matrix. For example, a company developing the device may decide the risk level, or range of risk levels, in the risk acceptability matrix that would be acceptable to the company based on the company's risk profile. Optionally, the risk management module 210 may request confirmation of the acceptability of the risk levels from the user. If the risk levels are determined to be acceptable, then the risk management module 210 may accept the update to the device design parameters, which may include updating the applicable document and/or databases.

If the risk management module 210 determines that the risks levels are not acceptable, then at block 818, the risk management module 210 identifies a design proposal to reduce the risk to an acceptable level. For example, returning to the above example with the cauterizer, the design proposal may include modifications to the energized component to improve its robustness such that the probability of damage when dropped is lowered, in turn lowering the risk of harm.

FIG. 9C includes a flowchart of a method for identifying comparable risks within an acceptable level, according to some embodiments. The flow 960 may be altered, e.g., by having one or more blocks added, removed, rearranged, combined, performed concurrently, and/or having one or more blocks split into multiple blocks.

At block 962, the risk management module 210 identifies one or more factors contributing to the risk levels. A factor may include a device design parameter or a combination of parameters for the device under development.

At block 964, the risk management module 210 determines the one or more factors with the greatest contribution (e.g., highest probabilities) to the risk levels.

At block 966, the risk management module 210 performs a cross-database search to identify comparable risks within an acceptable level, based on the cross-database relationships associated with the factor(s) with the greatest contribution to the risk levels.

At block 968, the risk management module 210 determines one or more device component(s) related to the comparable risks. For example, known device component(s) with a similar probability of the occurrence of the harm, and which has reduced the likelihood of harm occurring through potential risk control measures, may be determined to be components with comparable risks.

At block 970, the risk management module 210 generates the design proposal using the one or more device components(s) related to the comparable risks. The design proposal may include one or more updates to one or more attributes for the device under development in order to reduce the risks levels for the device to acceptable levels.

At block 972, the risk management module 210 displays the design proposal and requests confirmation of the updates to the one or more attributes.

Returning to FIG. 8, at block 820, the risk management module 210 determines whether a confirmation of the updates to the one or more attributes in the design proposal is received. If confirmation is not received, then the risk management module 210 returns to block 964 to further refine the attributes of the device until the risk levels are acceptable.

### Automatic Documentation and Real-Time Impact Assessment Module 212

The automatic documentation and real-time impact assessment module (“impact assessment module”) 212 manages the documentation processes, automatically performs impact assessments, and automatically compiles technical file documentation for medical device registration across different agencies and countries. In managing the documentation process, the impact assessment module 212 manages the device components and associated data points as design decisions are made through the platform 102. Each component of the device under development may include uniquely named components (e.g., “case,” “power supply”, “CPU”), where each component has one or more associated data points (e.g., “Material” with a value of “plastic” or “Color” with a value of “red”). The impact assessment module 212 may store data related to device components independently in customer-specific databases 228, ensuring that the data is available regardless of document creation.

Document templates are used to generate the documents, where the decision-data associations pulls the relevant document templates and populates the corresponding fields by pulling specific data values. Templates may be customized by the user for user-specific purposes and can be extended to include regulatory-specific templates (e.g., FDA-specific templates) using the same data structure.

An impact assessment is a required part of the document control process for any document change. In performing the impact assessment, the impact assessment module 212 obtains parameter definitions from users and embeds the parameters within a document according to regulatory requirements or user discretion. For regulatory requirements, the user may be prevented from removing the requirement from the document. The same element can be embedded multiple times within a single document or across multiple documents.

When a parameter's value is changed, the impact assessment module 212 queries other documents containing the same parameter. The impact assessment module 212 visualizes the impact of the change, allowing the user to confirm the updates. Upon confirmation, new versions of the affected documents are automatically generated with the updated element value.

Documents may be linked based on various dependencies, facilitating comprehensive impact assessments. These dependencies could be business rules, operation process requirement, medical device design control, FDA recommendations, etc. Changes to a parameter in one department can trigger cascading updates in related documents across different departments. The impact assessment module 212 identifies additional documents that need to be reviewed for potential scope changes based on these dependencies.

Each document is associated with specific roles, ensuring accountability and streamlined management. Dependency linking between documents allows for efficient retrieval and review of documents affected by scope changes in both upstream and downstream direction.

In compiling the technical file documentation, the impact assessment module 212 connects each design decision with the applicable documentation requirements. Based on the design decision, the impact assessment module 212 retrieves the applicable document template and creates a version of the document. Each design decision is recorded and saved as part of an audit trail, ensuring traceability and accountability. Regulatory submission forms are integrated within the platform 102. If a design decision is relevant to a submission form, the impact assessment module 212 automatically populates the forms with the recorded value. The submission forms may be broken down into parameters where the design decision is the value assigned to the parameter. For common decisions that apply to multiple region-specific forms, in response to a design decision, the impact assessment module 212 propagates the decision to the applicable forms. The impact assessment module 212 stores submission forms and validates the submission forms against official versions from the agencies. The official versions of the submission forms are continuously updated to reflect the latest design decisions and regulatory requirements and to ensure compliance with various regulatory agencies.

At the end of the design process, the submission forms can be exported for regulatory submission. The impact assessment module 212 integrates with regulatory agencies, providing users with specific dashboards and access to audit trails as part of the submission process.

FIG. 10 includes a flowchart of a method for automatic documentation and real-time impact assessment according to some embodiments. The flow 1000 is an example flow and not limiting. The flow 1000 may be altered, e.g., by having one or more blocks added, removed, rearranged, combined, performed concurrently, and/or having one or more blocks split into multiple blocks.

At block 1002, the impact assessment module 212 obtains one or more design decisions, with each design decision including one or more parameters and values for each parameter.

At block 1004, the impact assessment module 212 provides an impact assessment of the design decision. The impact assessment may include updates to be applied to existing downstream and/or upstream documents and/or new documents to be added to the current user's workflow or the workflow of another user due to the design decision.

At block 1006, the impact assessment module 212 requests confirmation of the impact assessment from the current user.

At block 1008, in response to receiving confirmation of the impact assessment from the current user, the impact assessment module 212 determines whether the impact assessment requires confirmation from other users. Such confirmation may be required when the impact assessment determines that the workflow of other users would be impacted by the design decision.

At block 1018, in response to determining that confirmation from other users is required, the impact assessment module 212 sends a request for confirmation to the other user(s).

At block 1020, the impact assessment module 212 determines whether confirmation of the impact assessment is received from the other user(s).

At block 1022, if the impact assessment module 212 fails to receive confirmation from the other user(s), then the impact assessment module 212 rolls back the document to a previous version.

If the impact assessment module 212 receives confirmation from the other users, then the impact assessment module 212 performs block 1010-1016.

At block 1010, the impact assessment module 212 stores the existing documents with the updated parameters and document versioning information in the databases 216.

At block 1012, the impact assessment module 212 adds any new documents to the workflow of one or more users.

At block 1014, the impact assessment module 212 removes any deleted documents from the workflow of one or more users.

At block 1616, the impact assessment module 212 updates the databases 216 with the relationships between the documents, relationships between the parameters in the documents, and the design decision.

At block 1024, the updates are propagated to the other modules in the platform 102.

### Generative AI Module 214

The GenIA module 214 may be configured to perform various functions with the other modules of the platform 102, as described. Further, using machine learning techniques, the GenAI module 214 may be configured to scrape documents, such as the 510k forms of the FDA, to identify relationships with predicate devices, standards and/or guidances, testing requirements, etc. The GenAI module 214 may then populate this information into a knowledge graph.

The knowledge graph may be generated using a variety of data sources from the FDA on medical device manufacturing and registration. These data sets may be used to create the entities of the graph, which include owners, establishments, US agents, official correspondents/applicants, devices, recalls, Unique Device Identifiers (UDIs), device classes, and product codes and their associated properties. The relationships between these entities include which entities are acting as agents or official correspondents to other entities, which entities have submitted which device for review, which devices have recalls or are associated with different classes and/or product codes, etc. Additionally, the knowledge graph may also contain data from the FDA on the technical contacts and specialty task groups associated with the relevant standards, FDA guidance, and regulations. Further, information about the applicable standards, such as referenced ISO standards, system requirements, subsystem requirements, specifications, and test requirements, may be contained within the graph.

The knowledge graph may be stored in a format that allows for information about the relationships between all entities (and their downstream relationships) to be queried.

The GenAI module 214 may further be configured to generate smart suggestions. For example, starting with a user-provided keyword (e.g., a product code, another 510(k) number, etc.), the GenAI module 214 may query the relationships in the knowledge graph to determine which other entities (devices, standards, testing requirements, etc.) might be related. The information may then be presented to a user in various ways. For example, the information may be displayed as a graph with axis linked to the one or more device design parameters. The graph may be dynamically updated based on a user selection of a parameter, such as a profile of a potential competitive company. Based on knowledge graph, the design input and output associated with a parameter may be presented as suggestions for a user to confirm.

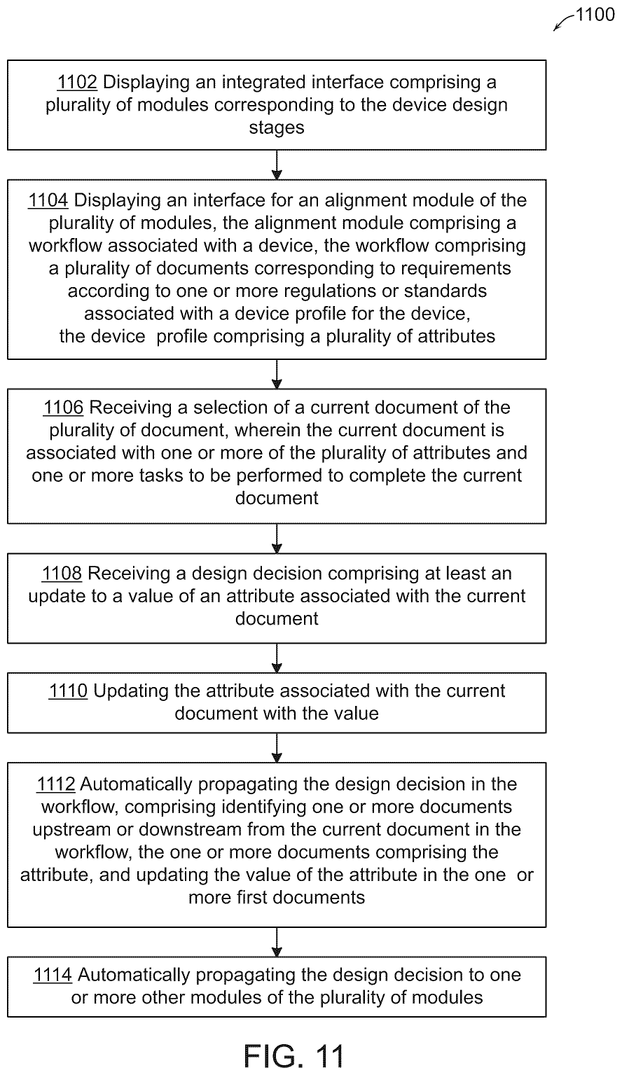

FIG. 11 includes a flowchart of a method for automatic alignment across device design stages in device development according to some embodiments. The flow 1100 is an example flow and not limiting. The flow 1100 may be altered, e.g., by having one or more stages added, removed, rearranged, combined, performed concurrently, and/or having one or more stages split into multiple stages.

At block 1102, the method 1100 includes displaying an integrated interface comprising a plurality of modules corresponding to the device design stages.

At block 1104, the method 1100 includes displaying an interface for an alignment module of the plurality of modules, the alignment module comprising a workflow associated with a device, the workflow comprising a plurality of documents corresponding to requirements according to one or more regulations or standards associated with a device profile for the device, the device profile comprising a plurality of attributes.

At block 1106, the method 1100 includes receiving a selection of a current document of the plurality of document, where the current document is associated with one or more of the plurality of attributes and one or more tasks to be performed to complete the current document.

At block 1108, the method 1100 includes receiving a design decision comprising at least an update to a value of an attribute associated with the current document.

At block 1110, the method 1100 includes updating the attribute associated with the current document with the value.

At block 1112, the method 1100 includes automatically propagating the design decision in the workflow, comprising identifying one or more documents upstream or downstream from the current document in the workflow, the one or more documents comprising the attribute, and updating the value of the attribute in the one or more first documents. For example, the propagating of the design decision in the workflow may further include identifying one or more second documents comprising at least another attribute whose value depends upon the value of the updated attribute, based on predetermined relationships between the plurality of attributes and the one or more second documents, and updating the other attribute in the one or more second documents based on the updated attribute. For another example, the propagating of the design decision in the workflow may further include determining one or more other documents to be added or deleted from the workflow due to the updated attribute, and updating the workflow to add or delete the one or more other documents.

At block 1114, the method 1100 includes automatically propagating the design decision to one or more other modules of the plurality of modules. For example, the propagating the design decision to one or more other modules of the plurality of modules may include displaying a traceability matrix, where the traceability matrix comprises a plurality of device design parameters for the device and a plurality of relationships between the plurality of device design parameters, and where the traceability matrix incorporates the updated attribute. The propagating of the design decision to one or more other modules may further include receiving a selection of one or more device design parameters in the traceability matrix, retrieving one or more other device design parameters associated with the selected one or more device design parameters, and determining one or more relationships between the selected one or more device design parameters and the retrieved one or more other device design parameters. The propagating of the design decision to one or more other modules may further include determining that the one or more relationships comprise one or more new or modified relationships and storing the one or more new or modified relationships.

The propagating of the design decision to one or more other modules may further include receiving a request for a context, displaying the context associated with the one or more relationships and an impact to the plurality of device design parameters upstream or downstream from the selected one or more device design parameters in one or more decision pathways, displaying a second design decision related to the given device design parameter in response to receiving a selection of a given device design parameter in the one or more decision pathways, and displaying the document in response to receiving a request to edit a document corresponding to the selected one or more device design parameters.

The propagating of the design decision to one or more other modules may further include determining that the traceability matrix comprises one or more orphan device design parameters, where each orphan parameter has no relationships to another device design parameter, receiving one or more modified or new relationships associated with the one or more orphan device design parameters, storing the one or more modified or new relationships with associations to the one or more orphan device design parameters.

For another example, propagating of the design decision to one or more other modules may include identifying one or more hazardous situation and foreseeable sequences of events applicable to the design decision, receiving a selection of an event of the one or more hazardous situation and foreseeable sequences of event, identifying one or more severity and occurrences of harm based on the selected event, receiving a selection of a harm of the one or more severity and occurrences of harm, calculating a current estimate of risk based on the selected harm, comparing the current estimate of risk with a risk acceptability matrix, and identifying a device design proposal that reduces the current estimate of risk in response to determining that the current estimate of risk fails to meet an acceptable level of risk.

For example, calculating of the current estimate of risk based on the selected harm and comparing the current estimate of risk with the risk acceptability matrix may further include calculating a residual risk as a delta between the current estimate of risk and a previous estimate of risk, and comparing the current estimate of risk and the residual risk with the risk acceptability matrix.

For example, identifying the one or more hazardous situation and foreseeable sequences of events applicable to the design decision may include performing a cross-database search to identify the one or more hazardous situation and foreseeable sequences of events applicable to a plurality of relationships between the plurality of device design parameters for the device, determining whether the one or more hazardous situation and foreseeable sequences of events comprise one or more gaps, generating a proposal comprising one or more missing hazardous situation and foreseeable sequences of events in response to determining that the one or more hazardous situation and foreseeable sequences of events comprise the one or more gaps, displaying the one or more hazardous situation and foreseeable sequences of events and the proposal, receiving the selection of the event from the one or more of the hazardous situation and foreseeable sequences of events and the proposal, and storing the selected event.

The identifying of the one or more severity and occurrences of harm based on the selected event may include performing a cross-database search to identify the one or more severity and occurrences of harm for the selected event based on cross-database relationships associated with the selected event, displaying the one or more identified severity and occurrences of harm, receiving the selection of harm from the one or more of the identified severity and occurrences of harm, and storing the selected harm. The identifying the device design proposal that reduces the current estimate of risk may include determining one or more factors contributing to the current estimate of risk, performing a cross-database search to identify one or more comparable risks with an acceptable level of risk based on cross-database relationships associated with the one or more factors, determining one or more device components of the device related to the one or more comparable risks, and generating the design proposal using the one or more device components of the device. The propagating of the design decision to one or more other modules of the plurality of modules may include determining that the identified one or more severity and occurrences of harm or the design proposal comprises an update to a second attribute of the plurality of attributes, and displaying one or more third documents in the workflow corresponding to the updated second attribute.

For another example, propagating of the design decision to one or more other modules may include providing an impact assessment of the design decision, where the impact assessment comprises one or more updates to one or more device design parameters for the device in one or more existing documents in the workflow, one or more new documents to be added to the workflow, or the one or more existing documents to be deleted from the workflow. The propagating of the design decision to one or more other modules may further include receiving confirmation of the impact assessment and updating the workflow based on the impact assessment in response to receiving the confirmation of the impact assessment. The updating of the workflow based on the impact assessment may include storing the one or more existing documents with the one or more updates to the one or more device design parameters with versioning information for the one or more existing documents, adding the one or more new documents to the workflow, or deleting the one or more existing documents from the workflow.

In an example embodiment, the method 1100 may further include receiving a change in one or more regulations applicable to the device, extracting information applicable to a plurality of device design parameters for the device from the one or more regulations, determining one or more relationships between the plurality of device design parameters based on the extracted information, and storing the one or more relationships between the plurality of device design parameters. The method 1100 may further include determining one or more impacts of the change in the one or more regulations to the plurality of device design parameters for the device and modifying the device profile and the workflow of the device based on the one or more impacts in response to receiving an acceptance of the one or more impacts.

In another example embodiment, the method 1100 may further include outputting a European Union Medical Device Regulation (EUMDR) regulatory submission by the platform, the EUMDR regulatory submission comprising: technical documentation comprising a design history file (DHF), a design master record (DMR), and post-market documents.

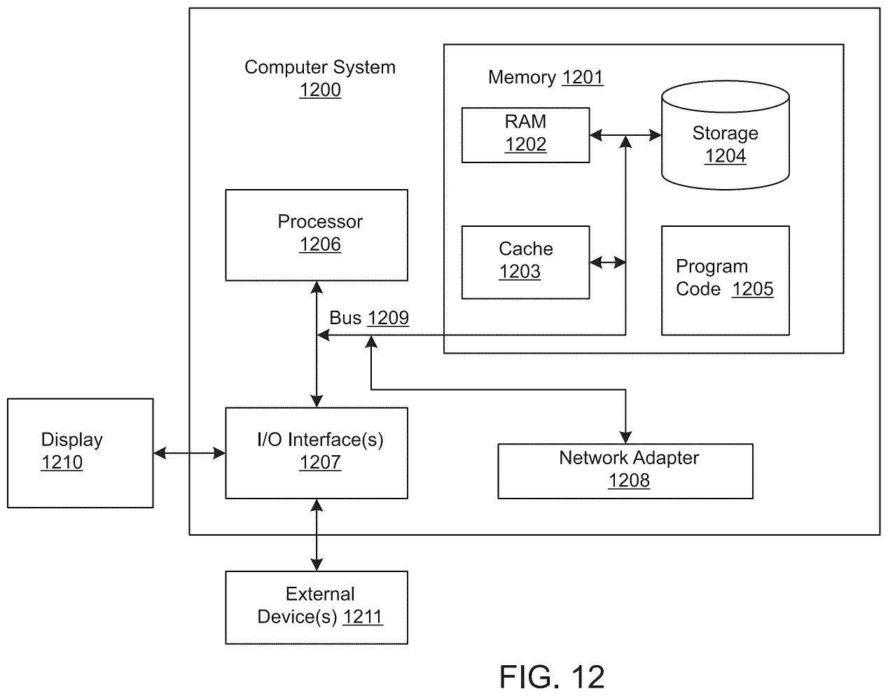

FIG. 12 includes a block diagram for a computer system according to some embodiments. The computer system 1200 may be an example of the server 100 or the client device 136 of FIG. 1. The computer system 1200 is operationally coupled to one or more processors or processing units 1206, one or more memories 1201, and a bus 1209 that couples various system components, including the one or more memories 1201 to the one or more processors 1206. The bus 1209 represents one or more of any of several types of bus structure, including a memory bus or memory controller, a peripheral bus, an accelerated graphics port, and a processor or local bus using any of a variety of bus architectures. The one or more memories 1201 may include computer readable media in the form of volatile memory, such as random access memory (RAM) 1202 or cache memory 1203, or non-volatile storage media 1204. The one or more memories 1201 may include at least one program product having a set of at least one program code module 1205 that are configured to carry out the functions of embodiment of the present invention when executed by the one or more processors 1206. The computer system 1200 may also communicate with one or more external devices 1211, such as a display 1210, via I/O interfaces 1207. The computer system 1200 may communicate with one or more networks via network adapter 1208.

### Other Considerations

Other examples and implementations are within the scope of the disclosure and appended claims. For example, due to the nature of software and computers, functions described above can be implemented using software executed by a processor, hardware, firmware, hardwiring, or a combination of any of these. Features implementing functions may also be physically located at various positions, including being distributed such that portions of functions are implemented at different physical locations.

As used herein, the singular forms “a,” “an,” and “the” include the plural forms as well, unless the context clearly indicates otherwise. Thus, reference to a device in the singular (e.g., “a device,” “the device”), including in the claims, includes at least one, i.e., one or more, of such devices (e.g., “a processor” includes at least one processor (e.g., one processor, two processors, etc.), “the processor” includes at least one processor, “a memory” includes at least one memory, “the memory” includes at least one memory, etc.). The phrases “at least one” and “one or more” are used interchangeably and such that “at least one” referred-to object and “one or more” referred-to objects include implementations that have one referred-to object and implementations that have multiple referred-to objects. For example, “at least one processor” and “one or more processors” each includes implementations that have one processor and implementations that have multiple processors. Also, a “set” as used herein includes one or more members, and a “subset”contains fewer than all members of the set to which the subset refers.

The terms “comprises,” “comprising,” “includes,” and/or “including,” as used herein, specify the presence of stated features, integers, steps, operations, elements, and/or components, but do not preclude the presence or addition of one or more other features, integers, steps, operations, elements, components, and/or groups thereof.

Also, as used herein, “or” as used in a list of items (possibly prefaced by “at least one of” or prefaced by “one or more of”) indicates a disjunctive list such that, for example, a list of “at least one of A, B, or C,” or a list of “one or more of A, B, or C” or a list of “A or B or C” means A, or B, or C, or AB (A and B), or AC (A and C), or BC (B and C), or ABC (i.e., A and B and C), or combinations with more than one feature (e.g., AA, AAB, ABBC, etc.). Thus, a recitation that an item, e.g., a processor, is configured to perform a function regarding at least one of A or B, or a recitation that an item is configured to perform a function A or a function B, means that the item may be configured to perform the function regarding A, or may be configured to perform the function regarding B, or may be configured to perform the function regarding A and B. For example, a phrase of “a processor configured to measure at least one of A or B” or “a processor configured to measure A or measure B” means that the processor may be configured to measure A (and may or may not be configured to measure B), or may be configured to measure B (and may or may not be configured to measure A), or may be configured to measure A and measure B (and may be configured to select which, or both, of A and B to measure).

As used herein, unless otherwise stated, a statement that a function or operation is “based on” an item or condition means that the function or operation is based on the stated item or condition and may be based on one or more items and/or conditions in addition to the stated item or condition.

Substantial variations may be made in accordance with specific requirements. For example, customized hardware might also be used, and/or particular elements might be implemented in hardware, software (including portable software, such as applets, etc.) executed by a processor, or both. Further, connection to other computing devices such as network input/output devices may be employed. Components, functional or otherwise, shown in the figures and/or discussed herein as being connected or communicating with each other are communicatively coupled unless otherwise noted. That is, they may be directly or indirectly connected to enable communication between them.

The systems and devices discussed above are examples. Various configurations may omit, substitute, or add various procedures or components as appropriate. For instance, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description herein to provide a thorough understanding of example configurations (including implementations). However, configurations may be practiced without these specific details. For example, well-known circuits, processes, algorithms, structures, and techniques have been shown without unnecessary detail in order to avoid obscuring the configurations. The description herein provides example configurations, and does not limit the scope, applicability, or configurations of the claims. Rather, the preceding description of the configurations provides a description for implementing described techniques. Various changes may be made in the function and arrangement of elements.

The terms “processor-readable medium,” “machine-readable medium,” and “computer-readable medium,” as used herein, refer to any medium that participates in providing data that causes a machine to operate in a specific fashion. Using a computing platform, various processor-readable media might be involved in providing instructions/code to processor(s) for execution and/or might be used to store and/or carry such instructions/code (e.g., as signals). In many implementations, a processor-readable medium is a physical and/or tangible storage medium. Such a medium may take many forms, including but not limited to, non-volatile media and volatile media. Non-volatile media include, for example, optical and/or magnetic disks. Volatile media include, without limitation, dynamic memory.

Having described several example configurations, various modifications, alternative constructions, and equivalents may be used. For example, the above elements may be components of a larger system, wherein other rules may take precedence over or otherwise modify the application of the disclosure. Also, a number of operations may be undertaken before, during, or after the above elements are considered. Accordingly, the above description does not bound the scope of the claims.

Unless otherwise indicated, “about” and/or “approximately” as used herein when referring to a measurable value such as an amount, a temporal duration, and the like, encompasses variations of ±20% or ±10%, ±5%, or ±0.1% from the specified value, as appropriate in the context of the systems, devices, circuits, methods, and other implementations described herein. Unless otherwise indicated, “substantially” as used herein when referring to a measurable value such as an amount, a temporal duration, a physical attribute (such as frequency), and the like, also encompasses variations of ±20% or ±10%, ±5%, or ±0.1% from the specified value, as appropriate in the context of the systems, devices, circuits, methods, and other implementations described herein.

A statement that a value exceeds (or is more than or above) a first threshold value is equivalent to a statement that the value meets or exceeds a second threshold value that is slightly greater than the first threshold value, e.g., the second threshold value being one value higher than the first threshold value in the resolution of a computing system. A statement that a value is less than (or is within or below) a first threshold value is equivalent to a statement that the value is less than or equal to a second threshold value that is slightly lower than the first threshold value, e.g., the second threshold value being one value lower than the first threshold value in the resolution of a computing system.

What is claimed is:

1. A computer-implemented method for automatic alignment across device design stages in device development, comprising:
displaying an integrated interface comprising a plurality of modules corresponding to the device design stages;
displaying an interface for an alignment module of the plurality of modules, the alignment module comprising a workflow associated with a device, the workflow comprising a plurality of documents corresponding to requirements according to one or more regulations or standards associated with a device profile for the device, the device profile comprising a plurality of attributes;
receiving a selection of a current document of the plurality of document, wherein the current document is associated with one or more of the plurality of attributes and one or more tasks to be performed to complete the current document;
receiving a design decision comprising at least an update to a value of an attribute associated with the current document;
updating the attribute associated with the current document with the value;
automatically propagating the design decision in the workflow, comprising identifying one or more documents upstream or downstream from the current document in the workflow, the one or more documents comprising the attribute, and updating the value of the attribute in the one or more first documents; and
automatically propagating the design decision to one or more other modules of the plurality of modules.

2. The method of claim 1, wherein automatically propagating the design decision in the workflow further comprises:
identifying one or more second documents comprising at least another attribute whose value depends upon the value of the updated attribute, based on predetermined relationships between the plurality of attributes and the one or more second documents; and
updating the other attribute in the one or more second documents based on the updated attribute.

3. The method of claim 1, wherein automatically propagating the design decision in the workflow further comprises:
determining one or more other documents to be added or deleted from the workflow due to the updated attribute; and
updating the workflow to add or delete the one or more other documents.

4. The method of claim 1, wherein automatically propagating the design decision to one or more other modules of the plurality of modules comprises:
displaying a traceability matrix, wherein the traceability matrix comprises a plurality of device design parameters for the device and a plurality of relationships between the plurality of device design parameters, wherein the traceability matrix incorporates the updated attribute;
receiving a selection of one or more device design parameters in the traceability matrix;
retrieving one or more other device design parameters associated with the selected one or more device design parameters; and
determining one or more relationships between the selected one or more device design parameters and the retrieved one or more other device design parameters.

5. The method of claim 4, wherein automatically propagating the design decision to one or more modules of the plurality of modules in the platform further comprises:
determining that the one or more relationships comprise one or more new or modified relationships; and
storing the one or more new or modified relationships.

6. The method of claim 4, wherein automatically propagating the design decision to one or more modules of the plurality of modules in the platform further comprises:
receiving a request for a context;
displaying the context associated with the one or more relationships and an impact to the plurality of device design parameters upstream or downstream from the selected one or more device design parameters in one or more decision pathways;
in response to receiving a selection of a given device design parameter in the one or more decision pathways, displaying a second design decision related to the given device design parameter; and
in response to receiving a request to edit a document corresponding to the selected one or more device design parameters, displaying the document.

7. The method of claim 4, wherein automatically propagating the design decision to the one or more other modules of the plurality of modules further comprises:
determining that the traceability matrix comprises one or more orphan device design parameters, wherein each orphan parameter has no relationships to another device design parameter;
receiving one or more modified or new relationships associated with the one or more orphan device design parameters; and
storing the one or more modified or new relationships with associations to the one or more orphan device design parameters.

8. The method of claim 1, wherein automatically propagating the design decision to the one or more other modules of the plurality of modules comprises:
identifying one or more hazardous situation and foreseeable sequences of events applicable to the design decision;
receiving a selection of an event of the one or more hazardous situation and foreseeable sequences of event;
identifying one or more severity and occurrences of harm based on the selected event;
receiving a selection of a harm of the one or more severity and occurrences of harm;
calculating a current estimate of risk based on the selected harm;
comparing the current estimate of risk with a risk acceptability matrix; and
identifying a device design proposal that reduces the current estimate of risk in response to determining that the current estimate of risk fails to meet an acceptable level of risk.

9. The method of claim 8, wherein calculating the current estimate of risk based on the selected harm and comparing the current estimate of risk with the risk acceptability matrix further comprise:
calculating a residual risk as a delta between the current estimate of risk and a previous estimate of risk; and
comparing the current estimate of risk and the residual risk with the risk acceptability matrix.

10. The method of claim 8, wherein identifying the one or more hazardous situation and foreseeable sequences of events applicable to the design decision comprises:
performing a cross-database search to identify the one or more hazardous situation and foreseeable sequences of events applicable to a plurality of relationships between the plurality of device design parameters for the device;
determining whether the one or more hazardous situation and foreseeable sequences of events comprise one or more gaps;
in response to determining that the one or more hazardous situation and foreseeable sequences of events comprise the one or more gaps, generating a proposal comprising one or more missing hazardous situation and foreseeable sequences of events;
displaying the one or more hazardous situation and foreseeable sequences of events and the proposal;
receiving the selection of the event from the one or more of the hazardous situation and foreseeable sequences of events and the proposal; and
storing the selected event.

11. The method of claim 10, wherein identifying the one or more severity and occurrences of harm based on the selected event comprises:
performing a cross-database search to identify the one or more severity and occurrences of harm for the selected event based on cross-database relationships associated with the selected event;
displaying the one or more identified severity and occurrences of harm;
receiving the selection of harm from the one or more of the identified severity and occurrences of harm; and
storing the selected harm.

12. The method of claim 11, wherein identifying the device design proposal that reduces the current estimate of risk comprises:
determining one or more factors contributing to the current estimate of risk;
performing a cross-database search to identify one or more comparable risks with an acceptable level of risk based on cross-database relationships associated with the one or more factors;
determining one or more device components of the device related to the one or more comparable risks; and
generating the design proposal using the one or more device components of the device.

13. The method of claim 12, wherein automatically propagating the design decision to one or more other modules of the plurality of modules further comprises:
determining that the identified one or more severity and occurrences of harm or the design proposal comprises an update to a second attribute of the plurality of attributes; and
displaying one or more third documents in the workflow corresponding to the updated second attribute.

14. The method of claim 1, wherein automatically propagating the design decision to the one or more other modules of the plurality of modules comprises:
providing an impact assessment of the design decision, wherein the impact assessment comprises: one or more updates to one or more device design parameters for the device in one or more existing documents in the workflow; one or more new documents to be added to the workflow; or the one or more existing documents to be deleted from the workflow;
receiving confirmation of the impact assessment; and
in response to receiving the confirmation of the impact assessment, updating the workflow based on the impact assessment.

15. The method of claim 14, wherein updating the workflow based on the impact assessment comprises:
storing the one or more existing documents with the one or more updates to the one or more device design parameters with versioning information for the one or more existing documents, adding the one or more new documents to the workflow, or deleting the one or more existing documents from the workflow.

16. The method of claim 1, further comprising:
receiving a change in one or more regulations applicable to the device;
extracting information applicable to a plurality of device design parameters for the device from the one or more regulations;
determining one or more relationships between the plurality of device design parameters based on the extracted information; and
storing the one or more relationships between the plurality of device design parameters.

17. The method of claim 16, further comprising:
determining one or more impacts of the change in the one or more regulations to the plurality of device design parameters for the device; and
in response to receiving an acceptance of the one or more impacts, modifying the device profile and the workflow of the device based on the one or more impacts.

18. The method of claim 1, further comprising:
outputting a Food and Drug Administration (FDA) regulatory submission by the platform, the FDA regulatory submission comprising: a design history record (DHR); a design master record (DRM); a digital data flow (DDF) file; and a medical device file (MDF) according to corresponding International Organization for Standardization (ISO) standards.

19. The method of claim 1, further comprising:
outputting a European Union Medical Device Regulation (EUMDR) regulatory submission by the platform, the EUMDR regulatory submission comprising: technical documentation comprising a design history file (DHF), a design master record (DMR), and post-market documents.

20. A system for automatic alignment across device design stages in device development, comprising:
one or more memories; and
one or more processors communicatively coupled to the one or more memories, the one or more processors being configured to:
display an integrated interface comprising a plurality of modules corresponding to the device design stages;
display an interface for an alignment module of the plurality of modules, the alignment module comprising a workflow associated with a device under development, the workflow comprising a plurality of documents corresponding to requirements according to one or more regulations or standards associated with a device profile for the device, the device profile comprising a plurality of attributes;
receive a selection of a current document of the plurality of document, wherein the current document is associated with one or more of the plurality of attributes and one or more tasks to be performed to complete the current document;
receive a design decision comprising at least an update to a value of an attribute associated with the current document;
update the attribute associated with the current document with the value;
automatically propagate the design decision in the workflow, comprising identifying one or more documents upstream or downstream from the current document in the workflow, the one or more documents comprising the attribute, and updating the value of the attribute in the one or more first documents; and
automatically propagate the design decision to one or more other modules of the plurality of modules.
